(19) United States
(12) Patent Application Publication
(10) Pub. No.: US 2020/0194101 A1
(43) Pub. Date: Jun. 18, 2020

(54) Methods and Systems for Monitoring Bacterial Ecosystems and Providing Decision Support for Antibiotic Use

(71) Applicant: Spokade Holdings Pty Ltd, Sydney (AU)

(72) Inventors: Guy Tsafnat, Sydney (AU); Sally Rachel Partridge, Sydney (AU)

(21) Appl. No.: 16/472,710

(22) PCT Filed: Jan. 8, 2018
(86) PCT No.: PCT/IB2018/000041

§ 371 Date: Jun. 21, 2019

Related U.S. Application Data
(60) Provisional application No. 62/444,222, filed on Jan. 9, 2017.

(51) Int. Cl. G16B 50/10; G16B 30/00; G16B 45/00; G16B 20/40; G16H 30/40

(52) U.S. Cl. CPC G16B 50/10; G16B 30/00; G16H 30/40; G16B 20/40; G16B 45/00

(57) ABSTRACT

The present disclosure provides computer-implemented methods for annotating a query nucleic acid sequence. Methods of the present disclosure provide for the accurate annotation of nucleic acid sequences having functional or other important implications. Subject methods also provide for generating an assembly for longer DNA sequences that comprise shorter annotated sequences. Also provided are methods for monitoring the genetic material within a defined physical location. Such methods may find use in a variety of applications, for example, monitoring the spread of a pandemic, monitoring the prevalence of antibiotic resistance, provide guidance in making clinical decisions, and others. Also provided are related systems and non-transitory computer-readable recording media.

### CROSS-REFERENCE

This application claims the benefit of U.S. Provisional Patent Application No. 62/444,222, filed Jan. 9, 2017, which application is incorporated herein by reference in its entirety.

### INTRODUCTION

Analysis of the genetic material obtained from a defined physical location can provide valuable information regarding organisms, e.g., pathogenic microorganisms, that are within a defined physical location. For example, the ability to identify the occurrence and/or frequency of specific antibiotic resistance genes within a defined physical location can provide information regarding the evolution of antibiotic resistance within the defined physical location, treatment options for a person in the defined physical location who is developing an infection, and others. Accordingly, there is a need in the art for improved methods of monitoring the genetic material within a defined physical location, including improved methods of annotating nucleic acid sequences originating from a defined physical location.

### SUMMARY

The present disclosure provides methods for annotating a query nucleic acid sequence obtained from a sample obtained from a defined physical location, which methods include accessing a relational database having a plurality of exemplar genetic elements and one or more fields associated with each exemplar genetic element.

For example, in a first embodiment, the present disclosure provides a computer-implemented method for annotating a query nucleic acid sequence, wherein the method includes the following steps performed by one or more computer processors: receiving a query nucleic acid sequence, wherein the query nucleic acid sequence is a sequence or segment thereof of a nucleic acid obtained from a sample obtained from a defined physical location; accessing a relational database including a plurality of exemplar genetic elements and the following fields associated with each exemplar genetic element: one or more identifying fields, an exemplar nucleic acid sequence for the exemplar genetic element or an identifier of the exemplar nucleic acid sequence, a minimum identity match criterion or identifier thereof, and an identifier for a matching algorithm.

The method further comprises receiving a selection of one or more of the exemplar genetic elements; for each of the selected one or more exemplar genetic elements, applying a corresponding matching algorithm identified in the identifier for a matching algorithm field to compare the query nucleic acid sequence with the exemplar nucleic acid sequence for the selected exemplar genetic element; for each of the selected one or more exemplar genetic elements, identifying whether results of the corresponding matching algorithm meet the minimum identity match criterion corresponding to the selected exemplar genetic element to provide a matched genetic element; for each matched genetic element, identifying whether constraints, if any, identified in the constraints identifier field corresponding to the selected exemplar genetic element have been met; and for one or more of the matched genetic elements without constraints and/or where the constraints corresponding to the selected exemplar genetic element have been met, annotating the query nucleic acid sequence with identifying information for the selected exemplar genetic element corresponding to the matched genetic element.

In a second embodiment, the present disclosure provides a method of monitoring the genetic material of a population of organisms in a defined physical location, wherein the method includes: obtaining nucleic acid sequences from a representative sample of the population of organisms from the defined physical location at one or more time points; annotating nucleic acid sequences from each of the representative samples according to a method of the first embodiment; and calculating a frequency of occurrence of a genetic element of interest in the population of organisms based on the annotation.

In a third embodiment, the present disclosure provides a method of monitoring the genetic material of a population of organisms in a defined physical location, wherein the method includes: collecting a representative sample of the population of organisms from the defined physical location at one or more time points; obtaining nucleic acid sequences from each of the representative samples; annotating the nucleic acid sequences according to the method of the first embodiment; and calculating a frequency of occurrence of a genetic element of interest in the population of organisms based on the annotation.

In a fourth embodiment, the present disclosure provides a method of monitoring the genetic material of a population of organisms in a defined physical location, wherein the method includes: collecting a representative sample of the population of organisms from the defined physical location at one or more time points; obtaining nucleic acid sequences from each of the representative samples; annotating the nucleic acid sequences by matching the nucleic acid sequences against a plurality of genetic elements in a relational database; and calculating a frequency of occurrence of a genetic element of interest in the population based on the annotation.

In a fifth embodiment, the present disclosure provides a method for obtaining an annotated nucleic acid sequence, wherein the method includes: inputting a query nucleic acid sequence via a client device over a network connection to a server device, wherein the server device performs the method according to the first embodiment to provide an annotated nucleic acid sequence; and receiving at the client device a representation of the annotated nucleic acid sequence.

In a sixth embodiment, the present disclosure provides a non-transitory computer-readable recording medium for annotating a query nucleic acid sequence, wherein the non-transitory computer-readable recording medium includes instructions, which, when executed by one or more processors, cause the one or more processors to perform a method for annotating a query nucleic acid sequence according to the first embodiment.

In a seventh embodiment, the present disclosure provides a non-transitory computer-readable recording medium for annotating a query nucleic acid sequence, wherein the non-transitory computer-readable recording medium includes instructions, which, when executed by one or more processors, cause the one or more processors to: receive a query nucleic acid sequence, wherein the query nucleic acid sequence is a sequence or segment thereof of a nucleic acid obtained from a sample obtained from a defined physical location; access a relational database comprising a plurality of exemplar genetic elements and the following fields associated with each exemplar genetic element: one or more identifying fields, an exemplar nucleic acid sequence for the exemplar genetic element or an identifier of the exemplar nucleic acid sequence, a minimum identity match criterion or identifier thereof, and an identifier for a matching algorithm.

The non-transitory computer-readable recording medium of the seventh embodiment further includes instructions, which, when executed by one or more processors, cause the one or more processors to: receive a selection of one or more of the exemplar genetic elements; for each of the selected one or more exemplar genetic elements, apply a corresponding matching algorithm identified in the identifier for a matching algorithm field to compare the query nucleic acid sequence with the exemplar nucleic acid sequence for the selected exemplar genetic element; for each of the selected one or more exemplar genetic elements, identify whether results of the corresponding matching algorithm meet the minimum identity match criterion corresponding to the selected exemplar genetic element to provide a matched genetic element; for each matched genetic element, identify whether constraints, if any, identified in the constraints identifier field corresponding to the selected exemplar genetic element have been met; and for one or more of the matched genetic elements without constraints and/or where the constraints corresponding to the selected exemplar genetic element have been met, annotate the query nucleic acid sequence with identifying information for the selected exemplar genetic element corresponding to the matched genetic element.

In an eighth embodiment, the present disclosure provides a system for annotating a query nucleic acid sequence, wherein the system includes: a communication module comprising an input manager for receiving the query nucleic acid sequence from a user; an output manager for communicating output to a user; and a non-transitory computer-readable recording medium according to the seventh embodiment.

The methods described herein may facilitate the discovery of, e.g., mobile elements and gene variants and may aid in monitoring the occurrence of pathogenic genetic elements in a defined physical location. Systems for practicing the subject methods are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 is a flow diagram of a method for annotating a query nucleic acid sequence, according to an example embodiment.

FIGS. 2A(a)-2A(c) depict how direct repeats are annotated, according to an example embodiment. FIGS. 2B(a)-2B(d) depict how reverse complement direct repeats are annotated, according to an example embodiment.

FIG. 3 is a flow diagram of a method for identifying and annotating a gap sequence within a query nucleic acid sequence, according to an example embodiment.

FIGS. 4A-4D depict different type of gap sequences that may be identified within a query nucleic acid sequence, according to example embodiments.

FIG. 5 is a flow diagram of a method for identifying and annotating a gap sequence within a query nucleic acid sequence, according to an example embodiment.

FIGS. 6A and 6B provide flow diagrams of a method for annotating a direct repeat on a query nucleic acid sequence, according to an example embodiment.

FIG. 7 is a flow diagram of a method for monitoring the frequency of occurrence of a genetic element of interest in a defined physical location, according to an example embodiment.

FIG. 8 is a flow diagram of a method for monitoring the frequency of occurrence of a genetic element of interest in a defined physical location, according to an example embodiment.

FIG. 9 is a block diagram of a system configured to carry out the subject methods, according to an example embodiment.

FIG. 10 is a block diagram of a system configured to carry out the subject methods, according to an example embodiment.

FIG. 11 is a flow diagram of the uses of a method of annotating a query nucleic acid sequence, according to example embodiments.

FIG. 12 is a flow diagram of a use of a method of annotating a query nucleic acid sequence, according to an example embodiment.

FIG. 13 is a flow diagram of a use of a method of annotating a query nucleic acid sequence, according to an example embodiment.

FIG. 14 is a flow diagram of the uses of a method of annotating a query nucleic acid sequence, according to example embodiments.

FIG. 15 is a flow diagram of the uses of a method of annotating a query nucleic acid sequence, according to example embodiments.

FIG. 16 is a sample relational database including various fields, according to an example embodiment.

FIGS. 17A and 17B depict an annotation image of exemplary annotation information for CP011639 (Serratia marcescens), according to an example embodiment.

### DETAILED DESCRIPTION

The present disclosure provides methods for annotating a query nucleic acid sequence obtained from a sample obtained from a defined physical location. The subject methods include accessing a relational database having a plurality of exemplar genetic elements and one or more fields associated with each exemplar genetic element. The methods described herein may facilitate the discovery of, e.g., mobile elements and gene variants and may aid in monitoring the occurrence of pathogenic genetic elements in a defined physical location. Systems for practicing the subject methods are also provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a nucleic acid sequence” includes a plurality of such nucleic acid sequences unless the context clearly dictates otherwise.

It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the disclosure or the definition or usage of any term herein conflicts with the disclosure or the definition or usage of any term in an application or publication incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The terms “nucleic acid”, “nucleic acid molecule”, “oligonucleotide” and “polynucleotide” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “nucleic acid sequence” refers to a contiguous string of nucleotide bases and in particular contexts also refer to the particular placement of nucleotide bases in relation to each other as they appear in an oligonucleotide. For example, the term “query nucleic acid sequence” refers to the nucleic acid sequence to be annotated by methods of the present disclosure. The term “exemplar nucleic acid sequence” is used to describe the nucleic acid sequence for an exemplar genetic element which is contained in a relational database used to annotate a query nucleic acid sequence.

The terms “polypeptide”, “amino acid sequence” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. For example, the term “query polypeptide”, “query protein” or “query amino acid sequence” refers to the amino acid sequence that may be annotated by methods of the present disclosure. Methods of the present disclosure may also be used to annotate amino acid sequences. The term “exemplar amino acid sequence” is used to describe the amino acid sequence for an exemplar peptide element which is contained in a relational database used to annotate a query amino acid sequence.

It should be noted that while the present disclosure focuses on the annotation of query nucleic acid sequences, the disclosed methods and systems may be readily adapted by one of skill in the art to the annotation of query polypeptide sequences, with the fields, constraints, etc., of the utilized databases adjusted accordingly.

As used herein, an “annotation” is a comment, explanation, note, link, descriptor, or the like, or a collection thereof, which may be applied to a nucleic acid sequence to characterize one or more features, e.g., one or more coding sequences, regulatory sequences, etc., of the nucleic acid sequence. Annotations may include pointers to external objects or external data. An annotation may optionally include information about an author who created or modified the annotation, as well as information about when that creation or modification occurred. For example, an annotation may be the act of assigning meaning to a query nucleic acid sequence, e.g. identifying segments of the query nucleic acid sequence as having a functional or a significant implication. Accurate annotation of a nucleic acid sequence may be used to identify, e.g., chromosomes, plasmids, mobile elements, specific regions of the nucleic acid sequence that uniquely identify a strain (e.g., a bacterial strain, a viral strain, etc.), virulence genes, specific gene variants of clinical and/or other significance, antibiotic resistance, etc.

As used herein, an “assembly” or “assembly of annotations” refers to a nucleic acid sequence that includes a collection of shorter annotated nucleic acid sequences. As will be apparent, annotation of partially assembled nucleic acid sequences can, e.g., reveal a mobile element present in the assembly that may be the result of recombination, and/or indicate regions in the assembly that may have multiple copies.

The term “genetic element” refers to a sequence of a nucleic acid sequence that represents, e.g., a gene, a genetic region, an insertion sequence, an inverted repeat, and the like. A mobile element (e.g., a mobile genetic element) refers to a genetic element or assembly that can move or code for a copy of itself that can move around within a cell and transpose itself into different locations in the same DNA molecule or in other DNA molecules. For example, a transposable element (e.g., an insertion sequence, a transposon, a retrotransposon, a DNA transposon, etc.), a plasmid, a genomic island, a bacteriophage, an intron, various viruses, and the like. Mobile elements may play a variety of clinically significant roles, for example, in the spread of virulence factors and antibiotic resistance. As used herein, an “exemplar genetic element” refers to a typical representation of a genetic element that can be used to annotate a nucleic acid sequence. An exemplar genetic element includes information used to identify the exemplar genetic element. An exemplar genetic element that has, e.g., met various criteria when compared to a nucleic acid sequence, provides for a matched genetic element, wherein the identifying information of the exemplar genetic element is used to annotate the matched genetic element within a query nucleic acid sequence.

As used herein, the terms “direct repeat”, “direct repeats” and the like, refer to a type of genetic sequence that includes two or more repeats of a specific nucleotide sequence. In some embodiments, the direct repeat is a nucleotide sequence present in multiple copies in the genome. In some embodiments, a direct repeat occurs when a sequence is repeated with the same pattern downstream, i.e., no inversion and/or no reverse complement is associated with the direct repeat. In some embodiments, direct repeats may have an intervening nucleotide sequence. Several types of repeated sequences are known in the art, for example: interspersed or dispersed DNA repeats (e.g., interspersed repetitive sequences) representing copies of transposable elements interspersed throughout a genome; flanking (or terminal) repeats representing sequences that are repeated on both ends of an intervening sequence (e.g., long terminal repeats on transposable elements), direct terminal repeats that are in the same direction, and reverse-complement terminal repeats that are in opposite directions relative to each other; and tandem repeats representing repeated copies that lie adjacent to each other, and may be direct or inverted tandem repeats.

A “direct repeat” may be a short sequences, e.g., a short sequence of from about 1 base pair (bp) to about 2 bp, e.g., from about 2 bp to about 4 bp, from about 3 bp to about 5 bp, from about 4 bp to about 6 bp, from about 5 bp to about 7 bp, from about 6 bp to about 8 bp, from about 7 bp to about 9 bp, from about 8 bp to about 10 bp, from about 9 bp to about 11 bp, from about 10 bp to about 12 bp, from about 11 bp to about 13 bp, from about 12 bp to about 14 bp, from about 13 bp to about 15 bp, from about 14 bp to about 16 bp, from about 15 bp to about 17 bp, from about 16 bp to about 18 bp, from about 17 bp to about 19 bp, from about 18 bp to about 20 bp, inclusive, that may be an artifact of a transposition of one or more insertion sequences, transposons, composite transposons and integrons.

As used herein, the term “database” refers generally to an organized collection of data stored in memory. In some embodiments, the database may be a relational database in which different tables and categories of the database are related to one another through at least one common attribute. In some embodiments, the database may include a server. In other embodiments, the term “database” may refer to computer software applications configured to interact with one or more client devices in order to analyze, capture, store, and process data. In other embodiments, the term “database” may refer to physical storage of data, such as hard disk storage. Or, in other embodiments, the term “database” may refer to a cloud-based storage system. Examples in industry include Google Drive and iCloud.

In some embodiments, a relational database of the present disclosure includes a plurality of exemplar genetic elements and various fields associated with each exemplar genetic element. Each field is generally associated with a value that provides information on how each field is interpreted by the relational database with respect to an exemplar genetic element. The value generally refers to a numerical value, and can, in some instances, refer to a symbol, text, nucleic acid sequence, or words. In some embodiments, a field includes an identifier of an algorithm associated with a particular exemplar genetic element which is to be applied in the context of the disclosed methods, e.g., an identifier for a matching algorithm. Fields of interest in connection with the disclosed methods include, but are not limited to, one or more identifying fields, which provide identifying information in connection with the exemplar genetic element; an exemplar nucleic acid sequence for the exemplar genetic element or an identifier of the exemplar nucleic acid sequence, e.g., an accession number or link to a nucleic acid sequence database; a minimum identity match criterion or identifier thereof, a directional identifier, a completeness identifier, a direct repeats identifier, and a constraints identifier.

The terms “system” and “computer-based system” refer to the hardware means, software means, and data storage means used to analyze the information of the present invention. Computer-based systems of the present disclosure may utilize the following hardware: a central processing unit (CPU), input means, output means, and data storage means. As such, any convenient computer-based system may be employed in the present invention. The data storage means may comprise any manufacture comprising a recording of the present information as described above, or a memory access means that can access such a manufacture.

A “processor” refers to any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer-readable recording medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory. A computer-readable recording medium may be a non-transitory computer-readable recording medium.

To “record” data, programming or other information on a computer readable medium refers to a process for storing information, using any convenient method. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

In certain embodiments, a system includes hardware components which take the form of one or more platforms, e.g., in the form of servers, such that any functional elements of the system, i.e., those elements of the system that carry out specific tasks (such as managing input and output of information, processing information, etc.) of the system may be carried out by the execution of software applications on and across the one or more computer platforms represented of the system. The one or more platforms present in the subject systems may be any convenient type of computer platform, e.g., such as a server, main-frame computer, a work station, etc. Where more than one platform is present, the platforms may be connected via any convenient type of connection, e.g., cabling or other communication system including wireless systems, either networked or otherwise. Where more than one platform is present, the platforms may be co-located or they may be physically separated. Various operating systems may be employed on any of the computer platforms, where representative operating systems include Windows, Sun Solaris, Linux, OS/400, Compaq Tru64 Unix, SGI IRIX, Siemens Reliant Unix, and others. The functional elements of system may also be implemented in accordance with a variety of software facilitators, platforms, or other convenient method.

As used herein, the term “remote location” is meant a location other than the location at which the referenced item is present. For example, a remote location could be another location (e.g., office, lab, etc.) in another part of the same room, another location in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

As described herein, a “client device” may refer to a personal computer, such as laptop, or also may refer to a mobile device or may refer to a computer tablet. Generally speaking, the client device refers to any hardware component including a processor or central processing unit (“CPU”) and a memory and a means of sending and receiving instructions. In some embodiments, the computer processor of the client device may be programmed to transmit and/or receive packets of data. In some embodiments, the client device may further include a data storage unit. In some embodiments, the client device may include a program, configured to execute instructions and/or receive instructions related to the process of annotating a query nucleic acid sequence. In some embodiments, the client device may include a non-transitory computer-readable recordable medium that includes a relational database for implementing the methods described herein.

As described above, the client device may be a first computing device or a component thereof. Alternatively, or in addition, a client device may include a second computing device or a component thereof. In some instances, the computing device may be a computer server. In some embodiments, the computing device may be a personal computer, tablet, and/or smartphone.

In some embodiments, the computer-implemented methods for annotating a query nucleic acid sequence can be implemented at least in part using structured query language (SQL). In some embodiments, the methods may be implemented at least in part using Hybrid-SQL instructions. In other embodiments, the methods may be implemented at least in part via NoSQL, xQuery, XPath, QUEL, MQL, LNQ. Any suitable query language that can be used to execute the methods described herein may be utilized in connection with such methods.

In some embodiments, the client device and/or relational database may include one or more computer processors. The one or more processors may execute instructions stored in the memory or storage of the client device and/or relational database. A program may cause one or more instructions to be executed in order to annotate a query nucleic acid sequence. In some embodiments, the program may be a web-based program. For example, web-based programs may be written with HTML or JavaScript or other web-native technologies that can be administered while the user is running a web browser over the internet.

As used in the claims, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising”, “consisting essentially of”, and “consisting of”, where one of these three terms is used herein, the presently disclosed subject matter can include the use of either of the other two terms.

### Methods

As summarized above, the present disclosure provides methods for annotating a query nucleic acid sequence. The subject methods include accessing a relational database having a plurality of exemplar genetic elements and one or more fields associated with each exemplar genetic element. The methods described herein may facilitate the discovery of, e.g., mobile elements and gene variants and may aid in monitoring the occurrence of pathogenic genetic elements in a defined physical location.

Methods for Annotating a Query Nucleic Acid Sequence

The present disclosure provides methods for annotating a query nucleic acid sequence (e.g., query DNA sequence). Methods of the present disclosure provide for the accurate annotation of nucleic acid sequences having functional or other important implications. Subject methods also provide for generating an assembly for longer DNA sequences that comprise shorter annotated sequences. In some embodiments, unique information can be obtained from the assembly, for example, the existence of mobile elements that may confer antibiotic resistance, virulence, and the like.

In some embodiments, a query nucleic acid sequence is a query DNA sequence. In some embodiments, a query nucleic acid sequence is a query RNA sequence. In some embodiments, a query nucleic acid sequence may be a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, primers, and the like. In some embodiments, a query nucleic acid sequence is a sequence or segment thereof of any of the above non-limiting examples of nucleic acids.

In some embodiments, a method of annotating a query nucleic acid sequence results in the query nucleic acid sequence being assigned a single annotation. In some embodiments, a method of annotating a query nucleic acid sequence results in the query nucleic acid sequence being assigned a plurality of annotations, for example, 2 annotations, 3 annotations, 4 annotations, 5 annotations, 6 annotations, 7 annotations, 8 annotations, 9 annotations, 10 annotations, 11 annotations, 12 annotations, 13 annotations, 14 annotations, 15 annotations, 20 annotations, 25 annotations, 30 annotations, 35 annotations, 40 annotations, 50 annotations, 60 annotations, 70 annotations, 80 annotations, or more. In such instances, the query nucleic acid sequence may be a longer nucleic acid sequence that includes several shorter nucleic acid sequences, each of which may be independently annotated. In some embodiments, a query nucleic acid sequence may include several non-overlapping annotations. In some embodiments, a query nucleic acid sequence may include several overlapping annotations. In such instances, the overlapping annotations may be fully overlapping, e.g., 100% overlapping, or may be partially overlapping, e.g., 5% overlapping, 10% overlapping, 15% overlapping, 20% overlapping, 25% overlapping, 30% overlapping, 35% overlapping, 40% overlapping, 45% overlapping, 50% overlapping, 55% overlapping, 60% overlapping, 65% overlapping, 70% overlapping, 75% overlapping, 80% overlapping, 85% overlapping, 90% overlapping, or 95% overlapping.

Of particular use in the methods described herein are query nucleic acid sequences, wherein the query nucleic acid sequences are sequences or segments thereof of nucleic acids obtained from a sample obtained from a defined physical location. As used herein, the term “defined physical location” refers to a defined area, space, or volume, e.g., a room, a surface, and the like. A defined physical location generally refers to an area that may be used for a specific purpose. For example, a defined physical location may be a residence, a bedroom, a hospital room, an operating room, a lab, an office, a restroom, a kitchen, a vehicle, etc., or a defined portion thereof. In some embodiments, a defined physical location is in a clinical setting. Non-limiting examples of defined physical locations in a clinical setting may include an emergency room, an operating room, an intensive care unit, a critical care unit, a hospital ward, a dispensary or pharmacy, an in-patient waiting room, an out-patient waiting room, a consulting room, a maternity ward, a laboratory, and the like, or a defined portion thereof. A defined physical location need not be an isolated room, and may be an area within a room, for example, a surface of any of the above non-limiting examples of defined physical locations (e.g., a waiting room chair, a hospital ward bed, a laboratory centrifuge, a wall of an emergency room, etc.).

Nucleic acids may be derived from a variety of sources. For example, nucleic acids may be derived from a bodily fluid. Non-limiting examples of bodily fluids include blood, saliva, sputum, feces, urine, amniotic fluid, breast milk, mucus, vomit, sweat, tears, ejaculate, puss and the like. In some embodiments, nucleic acids may be derived from eukaryotic cells (e.g., human cells), prokaryotic cells (e.g., bacterial cells), or viruses.

Accordingly, a method for annotating a query nucleic acid sequence includes receiving a query nucleic acid sequence, wherein the query nucleic acid sequence is a sequence or segment thereof of a nucleic acid obtained from a defined physical location. In general, a nucleic acid may be obtained from a defined physical location by various methods known in the art, for example, by swabbing a surface of the defined physical location. Any method known to those of skill in the art to purify and/or amplify a nucleic acid and to obtain the sequence or segment thereof of the nucleic acid may be used in connection with the disclosed methods and systems.

Relational Database:

The present disclosure provides computer-implemented methods for annotating a query nucleic acid sequence, wherein the methods include accessing a relational database that includes a plurality of exemplar genetic elements. For example, a method for annotating a query nucleic acid sequence may include steps performed by one or more computer processors, including: receiving a query nucleic acid sequence, and accessing a relational database.

A relational database of the present disclosure includes a plurality of exemplar genetic elements and various fields associated with each exemplar genetic element. Accordingly, the present disclosure includes methods for generating a relational database that includes a plurality of exemplar genetic elements and various fields (as described herein) associated with each exemplar genetic element. In some embodiments, the plurality of exemplar genetic elements is manually curated from experimental data. In some embodiments, the plurality of exemplar genetic elements is curated from one or more publicly available databases. In some embodiments, the plurality of exemplar genetic elements is generated from a combination of manual curations and curation from one or more publicly available databases. Non-limiting examples of publicly available databases include prokaryotic genome databases, e.g., Antibiotic Resistance Genes Database (ARDB), Bacillus subtilis Genome Database (BSORF and SubtiList), Chalmydomonas Resource Center, Database of E. coli mRNA Promoters with Experimentally Identified Transcriptional Start Sites (PromEC), E. coli Gene Expression Database (GenExpDB), Ensembl Bacteria, Escherichia coli Genome Database (Colibri), Horizontal Gene Transfer Database (HGT-DB), Human Microbiome Project (HMP), Interactive Atlas for Exploring Bacterial Genomes (BacMap), Microbial Genome Browser, Microbial Genome Database for Comparative Analysis (MBGD), Mycobacterium tuberculosis Genome (TubercuList), Operon Database (ODB), Prokaryotic Database of Gene Regulation (PRODORIC), and others; and mammalian genome databases, e.g., Encyclopedia of DNA Elements (ENCODE), Entrez Gene, Ensembl, GENCODE, Gene Ontology Consortium, GeneRIF, RefSeq, Uniprot, Vertebrate and Genome Annotation Project (VEGA), UCSC Genome Browser, GenBank, The Comprehensive Antibiotic Resistance Database (CARD), The ISfinder database, and others.

As discussed herein, in some embodiments, a relational database of the present disclosure includes a plurality of exemplar genetic elements and various fields associated with each exemplar genetic element. For example, a relational database may be in the format of a table, wherein each row of the relational database may represent an exemplar genetic element (e.g., a unique gene, sequence or segment thereof), and each column is represented by a field that provides information about the exemplary genetic element. Each field is generally associated with a value that provides information on how each field is interpreted by the relational database with respect to an exemplar genetic element. In some embodiments, a field includes an identifier of an algorithm associated with a particular exemplar genetic element which is to be applied in the context of the disclosed methods. The following are examples of fields that may be utilized in a relational database of the present disclosure.

Fields:

In some embodiments, a relational database includes one or more identifying fields, including for example: an identification (ID) field that provides a unique identifying number corresponding to the exemplary genetic element; a name field that provides an identifying name for the exemplary genetic element; a type field that provides information on the type of element the exemplary genetic element is (e.g., gene, genetic region, insertion sequence, inverted repeat, etc.); and the like.

In some embodiments, a relational database includes a sequence field that provides a nucleotide sequence of the exemplar genetic element. The sequence field provides an exemplar nucleic acid sequence for the exemplar genetic element or an identifier of the exemplar nucleic acid sequence, e.g., an accession number, or web link to a particular sequence in a sequence database. In some embodiments, the sequence may be a naturally occurring sequence (e.g., a DNA sequence, a RNA sequence, etc.). In some embodiments, the sequence may be a non-naturally occurring sequence, or may be a string of characters (e.g., a string of numerals, a string of letters, an alphanumeric string, etc.) that an appropriate algorithm can match a sequence of characters to. In some embodiments where the sequence is for example, a number, then the number is taken to be a reference to second exemplar genetic element. In such instances, the sequence and finder fields of the second exemplar genetic element are used for this exemplar genetic element (see, below for description relating to the finder field); and the minimum identity match and constraints fields are not taken from the second exemplar genetic element (see, below for description relating to the minimum identity match and constraints fields).

In some embodiments, a relational database includes a minimum identity match criterion (or identifier thereof) field that provides information on the degree or level of match the query nucleic acid sequence has to satisfy with respect to the nucleotide sequence of the exemplar genetic element, in order for the query nucleic acid sequence to be annotated with the exemplar genetic element. In some embodiments, the minimum identity match field provides a percentage value or criterion representing the degree or level of match the query nucleic acid sequence has to satisfy with respect to the nucleotide sequence of the exemplar genetic element, in order for the query nucleic acid sequence to be annotated with the exemplar genetic element. For example, the minimum identity match criterion may require the query nucleic acid sequence to match the nucleotide sequence of the exemplar genetic element with a sequence identity of a minimum of about 10%, a minimum of about 15%, a minimum of about 20%, a minimum of about 25%, a minimum of about 30%, a minimum of about 35%, a minimum of about 40%, a minimum of about 45%, a minimum of about 50%, a minimum of about 55%, a minimum of about 60%, a minimum of about 65%, a minimum of about 70%, a minimum of about 75%, a minimum of about 80%, a minimum of about 85%, a minimum of about 90%, a minimum of about 95%, a minimum of about 100%, in order for the query nucleic acid sequence to be annotated with the exemplar genetic element. In some embodiments, the minimum identity match criterion may be a sequence identity that ranges, e.g., from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 100%, from about 95% to about 100%, inclusive, in order for the query nucleic acid sequence to be annotated with the exemplar genetic element. As used herein, the term “sequence identity” refers the amount of characters (e.g., nucleotides) that match exactly between two different sequences (e.g., between the query nucleic acid sequence and the nucleotide sequence of the exemplar genetic element). In some embodiments, gaps within the sequences are not counted, and the measurement is relative to the shorter of the two sequences. The minimum identity match field provides a minimum identity match criterion or identifier thereof.

In some embodiments, a relational database includes a finder field that provides information on an appropriate algorithm for use with the nucleotide sequence of the exemplar genetic element. For example, the finder field may provide an identifier for a matching algorithm for use with the nucleotide sequence of the exemplar genetic element. The value presented in the finder field (e.g., name of a suitable matching algorithm) dictates how the sequence field and minimum identity match field is to be interpreted. Non-limiting examples of algorithms provided by a finder field include, e.g. a Strict Match algorithm that looks for the nucleotide sequence of the exemplar genetic element as a sub-sequence of the query nucleic acid sequence, a BLAST nucleotide similarity algorithm (as described in, e.g., Altschul, S. F. et al., Nucleic Acids Res. (1997) 25(17):3389-3402), a FASTA nucleotide similarity algorithm (as described in Pearson, W. R., et al., Proc. Natl. Acad. Sci. U.S.A. (1988) 85:2444-2448), a Smith-Waterman nucleotide similarity algorithm (as described in Smith, T. F. and Waterman, M. S., J. Mol. Biol. (1981) 147:195-197), a regular expression (RegEx) algorithm which uses a regular expression language to find matches (for example, as described in Myers, E. W. and Miller, W. Bull. Math. Biol. (1989) 51(1):5-37), and any other algorithms known to those of skill in the art for use in comparing nucleic acid sequences.

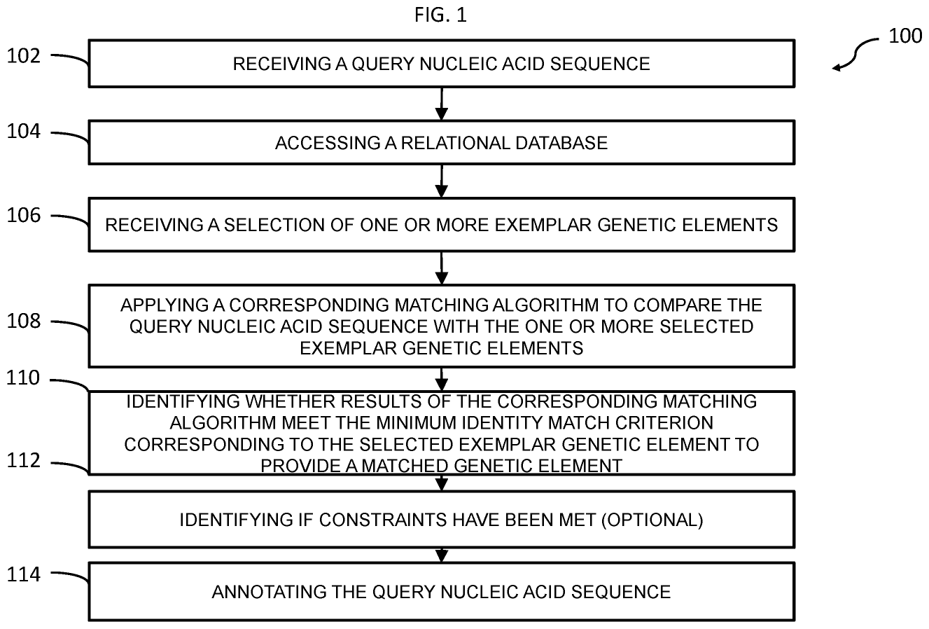

Accordingly, in some embodiments, a computer-implemented method for annotating a query nucleic acid sequence includes the following steps performed by one or more computer processors: receiving a query nucleic acid sequence, wherein the query nucleic acid sequence is a sequence or segment thereof of a nucleic acid obtained from a sample obtained from a defined physical location; accessing a relational database including a plurality of exemplar genetic elements and the following fields associated with each exemplar genetic element: one or more identifying fields, an exemplar nucleic acid sequence for the exemplar genetic element or an identifier of the exemplar nucleic acid sequence, a minimum identity match criterion or identifier thereof, and an identifier for a matching algorithm. FIG. 1 is a flow diagram of a method 100 for annotating a query nucleic acid sequence, according to an example embodiment. In step 102, a computer processor receives a query nucleic acid sequence. In step 104 a computer processor accesses a relational database, wherein the relational database includes a plurality of exemplar genetic elements and the following fields associated with each exemplar genetic element: one or more identifying fields, an exemplar nucleic acid sequence for the exemplar genetic element or an identifier of the exemplar nucleic acid sequence, a minimum identity match criterion or identifier thereof, and an identifier for a matching algorithm. In step 106, a computer processor receives a selection of one or more exemplar genetic elements contained within the relational database. It should be noted that step 106 can be performed before, after, or simultaneously with step 104. In step 108, a matching algorithm identified in the identifier for a matching algorithm field corresponding to each of the selected one or more exemplar genetic elements is applied to compare the query nucleic acid sequence with the one or more selected exemplar genetic elements, respectively. In step 110, for each of the selected one or more exemplar genetic elements, a computer processor identifies whether results of the corresponding matching algorithm meet the minimum identity match criterion corresponding to the selected exemplar genetic element to provide a matched genetic element. Step 112 includes identifying whether constraints, if any, identified in the constraints identifier field corresponding to the selected exemplar genetic element have been met. It should be noted that the constraints identifier field is optional in the relational database and may be excluded in suitable embodiments. In step 114, the query nucleic acid sequence is annotated with identifying information of any matched genetic element, which either meets the constraints corresponding to the selected exemplar genetic element or for which constraints are not present.

In some embodiments, a relational database includes a directional field that provides information about whether the direction of the nucleotide sequence of the exemplar genetic element should be considered or not in the annotation. The directional field provides a directional identifier that dictates whether the direction of the nucleotide sequence of the exemplar genetic element should be considered or not in the annotation. For example, in some embodiments, if the value for the directional field is ‘true’, then the exemplar genetic element is always to be treated in the annotation relative to the direction implied by the nucleotide sequence of the exemplar genetic element. In other embodiments, if the value of the directional field is ‘false’ then the direction of the nucleotide sequence of the exemplar genetic element is not taken into consideration in the annotation. Accordingly, the value for the directional identifier field for the selected exemplar genetic element corresponding to the matched genetic element (as described below) indicates whether the direction of the corresponding exemplar nucleic acid sequence should be noted in the corresponding annotation of the query nucleic acid sequence.

In some embodiments, a relational database includes a partial field that provides information on whether the nucleotide sequence for the exemplar genetic element represents a complete or incomplete nucleotide sequence of the exemplar genetic element. In some embodiments, the partial field provides a completeness identifier that indicates whether the nucleotide sequence for the exemplar genetic element represents a complete or incomplete nucleotide sequence of the exemplar genetic element. Accordingly, a match to such an exemplar genetic element may be annotated as partial. In some embodiments, the partial field provides a NOT-PARTIAL or a PARTIAL-ONLY constraint. A NOT-PARTIAL constraint indicates that the exemplar genetic element should only be matched in its entirety, and no annotation of partial features is allowed. For example, in some embodiments, a relational database includes a not-partial field that provides information on whether a query nucleic acid sequence that matches the nucleotide sequence of an exemplar genetic element is considered only if the complete nucleotide sequence of the exemplar genetic element is found within the query nucleic acid sequence. A PARTIAL-ONLY constraint indicates that the exemplar genetic element should only be matched as an annotation of part of the exemplar genetic element, and never in its entirety. Accordingly, the value for the partial field for the selected exemplar genetic element corresponding to the matched genetic element (as described below) indicates whether (a) the exemplar nucleic acid sequence for the exemplar genetic element is a complete or incomplete sequence for the selected exemplar genetic element (and the query nucleic acid sequence is annotated accordingly if matched), (b) whether the exemplar genetic element should only be matched in its entirety, or (c) whether the exemplary genetic element should only be matched in part.

In some embodiments, a relational database includes an alert field that provides information of when, if at all, an alert should be raised if a particular exemplar genetic element is found in the query nucleic acid sequence. The alert field provides an alert identifier that raises an alert when the associated exemplar genetic element is used to annotate the query nucleic acid sequence. Variations on the value for the alert field dictate various outcomes. For example, in some embodiments, if the alert field is set to ‘no’, then an alert is not raised when the associated exemplar genetic element is used to annotate the query nucleic acid sequence. In other embodiments, if the alert field is set to ‘complete’ then an alert is raised if the complete nucleotide sequence of the associated exemplar genetic element is used to annotate the query nucleic acid sequence. In other embodiments, if the alert field is set to ‘any’ then an alert is raised if the complete nucleotide sequence of the associated exemplar genetic element, or a segment thereof, is used to annotate the query nucleic acid sequence.

In some embodiments, a relational database includes a direct repeats field that provides information on whether the nucleotide sequence of an exemplar genetic element includes a direct repeat. The direct repeats field provides a direct repeats identifier that indicates whether the nucleotide of the exemplar genetic element includes a direct repeat.

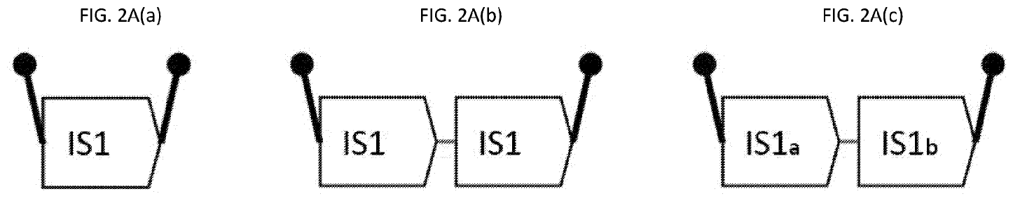

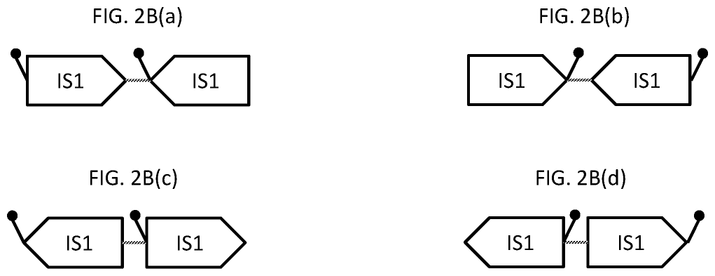

For example, certain mobile elements (e.g., IS1, IS26) replicate short sequences during their self-integration into a target nucleic acid sequence. Such elements may be found in wild-type DNA flanked by direct repeats. Referring to FIGS. 2A-2C, black ‘lollipops’ indicate direct repeat annotations and a pentagon indicates a mobile element annotation (e.g., an insertion sequence (e.g., IS1)) (FIG. 2A). In some cases, direct repeats may flank a segment that starts and ends in two copies of the nucleotide sequence of an exemplar genetic element (FIG. 2B). In some cases, a gap in the annotation may occur (represented by horizontal line between the two pentagons of FIG. 2B). In some cases, direct repeats can occur between non-identical nucleotide sequences of exemplar genetic elements (represented by “IS1a” and “IS1b” in FIG. 2C).

The length of direct repeats may vary depending on the exemplar genetic element. For example, a direct repeat may be a short sequence of from about 1 base pair (bp) to about 2 bp, e.g., from about 2 bp to about 4 bp, from about 3 bp to about 5 bp, from about 4 bp to about 6 bp, from about 5 bp to about 7 bp, from about 6 bp to about 8 bp, from about 7 bp to about 9 bp, from about 8 bp to about 10 bp, from about 9 bp to about 11 bp, from about 10 bp to about 12 bp, from about 11 bp to about 13 bp, from about 12 bp to about 14 bp, from about 13 bp to about 15 bp, from about 14 bp to about 16 bp, from about 15 bp to about 17 bp, from about 16 bp to about 18 bp, from about 17 bp to about 19 bp, from about 18 bp to about 20 bp, inclusive. In some embodiments, the length of direct repeats is constant. In such instances, the length of the expected direct repeat may be recorded in the direct repeats field as an integer representing the number of nucleotides repeated. In some embodiments, the number of direct repeats may be variable, and in some cases, within a constraint range. In such instances, the number of direct repeats may be recorded in the direct repeats field as a range of two integers. For example, if the number of direct repeats associated with the exemplar genetic element is expected to be within the range of 5 to 8 repeats, then the range of 5-8 may be recorded in the direct repeats field. In some embodiments, the nucleotide sequences of exemplar genetic elements may form direct repeats with each other. In such instances, the possible pairs of direct repeats can be recorded in the direct repeats field using the keyword ‘WITH’. For example “5 with ‘IS1’, ‘IS1a’, ‘IS1b’” may be recorded in the direct repeats field indicating that direct repeats may form between the exemplar genetic elements IS1, IS1a and IS1b. Accordingly, the value for the direct repeats identifier field for the selected exemplar genetic element corresponding to the matched genetic element (as described below) indicates whether the exemplar nucleic acid sequence for the exemplar genetic element includes direct repeats.

In some embodiments, a relational database includes a constraints field that provides additional information that is part of the exemplar genetic element. The constraints field provides a constraints identifier that indicates any additional criteria that is to be applied to the exemplar genetic element in order for the query nucleic acid sequence to be annotated with the exemplar genetic element. Constraints are applied, when present, to a query nucleic acid sequence that the finder has already identified as matching the nucleotide sequence of the exemplar genetic element. Various constraints may be applied including, for example, an open reading frame (ORF) constraint, a specific nucleotide constraint, a length constraint, or a combination of constraints combined using Boolean operators (e.g., AND, OR and NOT). In embodiments where a combination of constraints are applied to a query nucleic acid sequence that the finder has already identified as matching the nucleotide sequence of the exemplar genetic element, parentheses can be used in the field to indicate precedence and nesting.

In some embodiments, an open reading frame (ORF) constraint may be applied to a query nucleic acid sequence that the finder has already identified as matching the nucleotide sequence of the exemplar genetic element. The ORF constraint identifies a particular amino acid sequence that has to be derived from the query nucleic acid sequence and has to match exactly with the amino acid sequence of the exemplar genetic element as given in the constraint. In some embodiments, an ORF constraint follows the general format of ORF n-m ‘AMINO ACID SEQUENCE’, where ORF is the keyword that identifies the type of constraint to be applied, n and m are positions within the exemplar genetic element's nucleotide sequence that correspond to the open reading frame that is to be translated, and AMINO ACID SEQUENCE is the amino acid sequence that should be translated from the indicated open reading frame. In some cases, if n is omitted, it can be replaced with the value 1. In some cases, if m is omitted, the value for m can be calculated from the amino acid sequence. For example, if the query nucleic acid sequence to be annotated must have a nucleotide sequence between positions 17 and 40 (inclusive) that translates to the amino acid sequence “MRISLALC”, the below may be input into the constraints field.

### ORF 17-40 ‘MRISLALC’

In some embodiments, a specific nucleotide constraint may be applied to a query nucleic acid sequence that the finder has already identified as matching the nucleotide sequence of the exemplar genetic element. The specific nucleotide constraint indicates that at specific positions, certain nucleotides have to be found within the query nucleic acid sequence that has been identified as matching the nucleotide sequence of the exemplar genetic element. In some embodiments, a specific nucleotide constraint follows the general format of AT n HAS ‘b’, where n is a position relative to the start of the nucleotide sequence of the exemplar genetic element and b is a nucleotide character (e.g., one of a, c, g or t). A nucleotide character can also be represented by, e.g., n when the nucleotide is one of a, c, g or t; b when the nucleotide is one of c, g or t; d when the nucleotide is one of a, g or t; h when the nucleotide is one of a, c or t; v when the nucleotide is one of a, c or g; r when the nucleotide is one of a or g; y when the nucleotide is one of c or t; m when the nucleotide is one of a or c; k when the nucleotide is one of g or t; s when the nucleotide is one of c or g, w when the nucleotide is one of a or t; and in some embodiments, u may represent t. For example, if the query nucleic acid sequence to be annotated must have a ‘g’ at position 129 of the nucleotide sequence of the exemplar genetic element, the below may be input into the constraints field.

AT 129 HAS ‘g’

In some embodiments, a length constraint may be applied to a query nucleic acid sequence that the finder has already identified as matching the nucleotide sequence of the exemplar genetic element. The length constraint indicates a minimum or maximum length, or a range, that is required of the query nucleic acid sequence that has been identified as matching the nucleotide sequence of the exemplar genetic element. In some embodiments, a length constraint follows the general format of LENGTH Op n, where LENGTH is the keyword indicating that a length constraint is to be applied, n is an integer, and Op is one of the following relational operators: =(equal to), !=(not equal to), > (greater than), >=(greater than or equal to), <(less than), and <=(less than or equal to). For example, if the query nucleic acid sequence to be annotated must have at least 300 nucleotides that match to the nucleotide sequence of the exemplar genetic element, the below may be input into the constraints field.

### LENGTH >=300

In some embodiments, a combination of constraints may be applied to a query nucleic acid sequence that the finder has already identified as matching the nucleotide sequence of the exemplar genetic element. In such instances, the combination of constraints may be combined using Boolean operators (e.g., AND, OR and NOT). In embodiments where a combination of constraints are applied to a query nucleic acid sequence that the finder has already identified as matching the nucleotide sequence of the exemplar genetic element, parentheses can be used in the field to indicate precedence and nesting. For example, if the query nucleic acid sequence to be annotated must have at least 300 nucleotides that match to the nucleotide sequence of the exemplar genetic element, and have a ‘g’ or an ‘a’ at position 27 of the nucleotide sequence of the exemplar genetic element, the below may be input into the constraints field. In some embodiments, the constraint that is entered into a field is case-sensitive. In some embodiments, the constraint that is entered into a field is case-insensitive.

LENGTH >=300 AND (AT 27 HAS ‘g’ OR AT 27 HAS ‘a’)

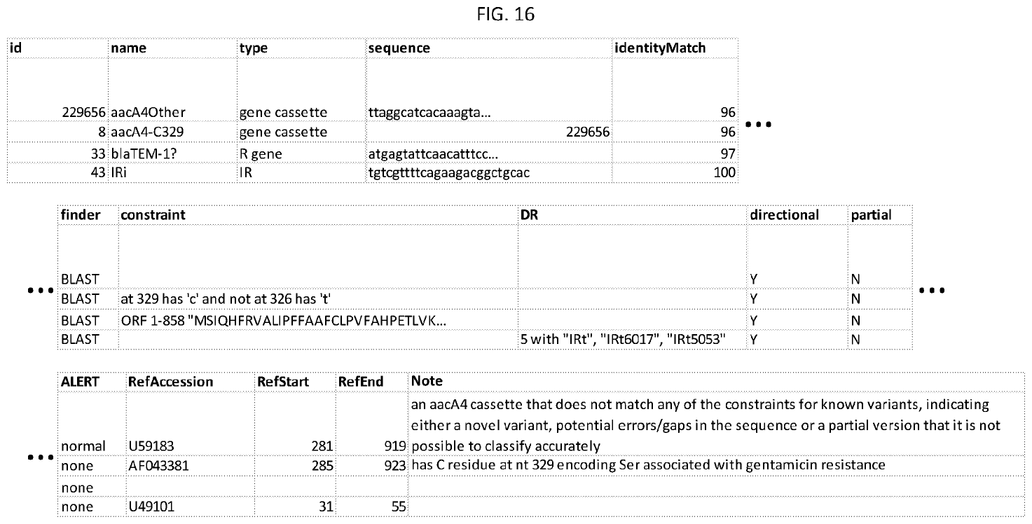

FIG. 16 provides an embodiment of a sample relational database containing various fields including, id (identification), name, type, sequence, identityMatch (e.g., minimum identity match), finder (e.g., matching algorithm), constraint, DR (direct repeats), directional, partial, ALERT, RefAccession (reference accession number), RefStart (position at which the reference sequence begins), RefEnd (position at which the reference sequence ends), and note (for any notes regarding the exemplar genetic element).

Accordingly, a computer-implemented method for annotating a query nucleic acid sequence includes the following steps performed by one or more computer processors: receiving a query nucleic acid sequence, wherein the query nucleic acid sequence is a sequence or segment thereof of a nucleic acid obtained from a sample obtained from a defined physical location; accessing a relational database having a plurality of exemplar genetic elements and various fields associated with each exemplar genetic element, wherein the various fields include, for example: one or more identifying fields, a sequence field that provides an exemplar nucleic acid sequence for the exemplar genetic element or an identifier of the exemplar nucleic acid sequence, a minimum identity match field that provides a minimum identity match criterion or identifier thereof, an identifier for a matching algorithm, a directional identifier, a completeness identifier, a direct repeats identifier, a constraints identifier and an alert identifier. In some embodiments, a computer-implemented method for annotating a query nucleic acid sequence comprises the following steps performed by one or more computer processors: receiving a query nucleic acid sequence, wherein the query nucleic acid sequence is a sequence or segment thereof of a nucleic acid obtained from a sample obtained from a defined physical location; accessing a relational database comprising a plurality of exemplar genetic elements and the following fields associated with each exemplar genetic element: one or more identifying fields, an exemplar nucleic acid sequence for the exemplar genetic element or an identifier of the exemplar nucleic acid sequence, a minimum identity match criterion or identifier thereof, an identifier for a matching algorithm, a directional identifier, a completeness identifier, a direct repeats identifier, an alert identifier, and a constraints identifier; wherein the constraints identifier corresponds to a constraint comprising an open reading frame constraint, a specific nucleotide constraint, a length constraint, or a combination thereof.

In some embodiments, a relational database optionally includes additional fields that may add valuable information to the annotation process. Additional fields may include an alternative names field indicating alternative names by which the exemplar genetic element may be known, a reference accession field indicating a hyperlink to a public repository (e.g., GenBank) that comprises an exemplar nucleotide sequence of the exemplar genetic element, a reference start field indicating the starting position of the nucleotide sequence of the exemplar genetic element in the query nucleic acid sequence, a reference end field indicating the ending position of the nucleotide sequence of the exemplar genetic element in the query nucleic acid sequence, and a notes field indicating any comments about the exemplar genetic element, including how to cite its annotation in the query nucleic acid sequence.

In some embodiments, a relational database includes a constraint field. In some embodiments, a relational database includes a constraint field and a direct repeats field. In some embodiments, a relational database includes a constraint field, a direct repeats field, and a minimum identity match field. In some embodiments, a relational database includes a constraint field, a direct repeats field, a minimum identity match field, and a finder field. In some embodiments, a relational database includes a constraint field, a direct repeats field, a minimum identity match field, a finder field, and a partial field. In some embodiments, a relational database includes a constraint field, a direct repeats field, a minimum identity match field, a finder field, a partial field, and a directional field.

Those of skill in the art will be able to select the suitable fields required in a relational database used for annotating a query nucleic acid sequence. The above fields are to be taken as exemplary fields that a relational database may include, and are to be taken as a non-limiting list of fields that may be selected from. Additional fields that may be included in a relational database for annotating a query nucleic acid sequence will be apparent to one of skill in the art, and one of skill in the art will be able to add and implement additional fields to the relational database.

Methods of Annotation:

The present disclosure provides computer-implemented methods for annotating a query nucleic acid sequence. For example, a method for annotating a query nucleic acid sequence according to the present disclosure may include steps performed by one or more computer processors, including: receiving a query nucleic acid sequence, wherein the query nucleic acid sequence is a sequence or segment thereof of a nucleic acid obtained from a sample obtained from a defined physical location, accessing a relational database that includes a plurality of exemplar genetic elements, and receiving a selection of one or more of the exemplar genetic elements.

In some embodiments, the relational database includes a plurality of exemplar genetic elements, and all of the exemplar genetic elements are selected for use in annotating a query nucleic acid sequence. In some embodiments, a subset of the exemplar genetic elements is selected for use in annotating a query nucleic acid sequence. The subset or selection of exemplar genetic elements used in annotating a query nucleic acid sequence depends on the type of query nucleic acid sequence to be annotated. Those of skill in the art will be able to decide whether the whole plurality of exemplar genetic elements included in the relational database will be used, or a subset or selection of the plurality of exemplar genetic elements will be used to annotate a query nucleic acid sequence of interest.

Accordingly, in some embodiments, a computer-implemented method for annotating a query nucleic acid sequence includes the following steps performed by one or more computer processors: receiving a query nucleic acid sequence, wherein the query nucleic acid sequence is a sequence or segment thereof of a nucleic acid obtained from a sample obtained from a defined physical location; accessing a relational database comprising a plurality of exemplar genetic elements (and including various field associated with each exemplar genetic element as described above); and receiving a selection of one or more of the exemplar genetic elements. In some embodiments, for each of the selected one or more exemplar genetic elements, the method further includes applying a corresponding matching algorithm identified in the identifier for a matching algorithm field to compare the query nucleic acid sequence with the exemplar nucleic acid sequence for the selected exemplar genetic element.

In some embodiments, each of the selected one or more exemplar genetic elements is compared, using its corresponding matching algorithm indicated in the finder field of the relational database, to the query nucleic acid sequence with the nucleotide sequence of the exemplar genetic element. Suitable matching algorithms are described above, but may include a Strict Match algorithm, a FASTA algorithm, a Smith-Waterman algorithm, a Regular Expression (RegEx) algorithm, or any suitable matching algorithm known to those of skill in the art.

In some embodiments, for each of the selected one or more exemplar genetic elements, a method for annotating a query nucleic acid sequence further includes identifying whether results of the corresponding matching algorithm meet the minimum identity match criterion corresponding to the selected exemplar genetic element. Each of the selected one or more exemplar genetic elements that meet the minimum identity match criterion corresponding to the selected exemplar genetic element provides a matched genetic element. In other words, a matched genetic element is an exemplar genetic element in which results of the corresponding matching algorithm for the exemplar genetic element has met the minimum identity match criterion corresponding to the exemplar genetic element. In some embodiments, the matching algorithm corresponding to the exemplar genetic element allocates a start and end position of any nucleic acid sequence or segments thereof that match the exemplar genetic element. In such instances, the start and end positions are relative to the start and end of the query nucleic acid sequence being annotated. In some embodiments, the matching algorithm may calculate a matching algorithm score indicating how well the corresponding exemplar genetic element and the query nucleic acid sequence match. The calculated matching algorithm score indicates the level of match between the query nucleic acid sequence or segment thereof and the matched genetic element.

In some embodiments, the step of generating matched genetic elements may be performed on multiple computers, each with its own copy of the query nucleic acid sequence to be annotated. In such instances, the step of generating matched genetic elements may be performed on multiple computers in parallel and may be used to monitor the consistency of match results and may improve the accuracy in annotating a query nucleic acid sequence. In some embodiments, the step of generating matched genetic elements may be performed on one or more, two or more, three or more, four or more, five or more, six or more, seven or more, eight or more, nine or more, ten or more computers operating in parallel.

In some embodiments, for each matched genetic element, the method for annotating a query nucleic acid sequence further includes identifying whether constraints, if any, identified in the constraints identifier field (see, description of the constraints field above) corresponding to the selected exemplar genetic element have been met. In such instances, a query nucleic acid sequence is annotated with identifying information of an exemplar genetic element if the matching algorithm corresponding to the exemplar genetic element provides results that meet the minimum identity match criterion and the query nucleic acid sequence has passed all, if any, of the constraints corresponding to the exemplar genetic element.

Accordingly, in some embodiments, a computer-implemented method for annotating a query nucleic acid sequence includes the following steps performed by one or more computer processors: receiving a query nucleic acid sequence, wherein the query nucleic acid sequence is a sequence or segment thereof of a nucleic acid obtained from a sample obtained from a defined physical location; accessing a relational database comprising a plurality of exemplar genetic elements and various fields associated with each exemplar genetic element; receiving a selection of one or more of the exemplar genetic elements; for each of the selected one or more exemplar genetic elements, applying a corresponding matching algorithm identified in the identifier for a matching algorithm field to compare the query nucleic acid sequence with the exemplar nucleic acid sequence for the selected exemplar genetic element; for each of the selected one or more exemplar genetic elements, identifying whether results of the corresponding matching algorithm meet the minimum identity match criterion corresponding to the selected exemplar genetic element to provide a matched genetic element; for each matched genetic element, identifying whether constraints, if any, identified in the constraints identifier field corresponding to the selected exemplar genetic element have been met; and for one or more of the matched genetic elements without constraints and/or where the constraints corresponding to the selected exemplar genetic element have been met, annotating the query nucleic acid sequence with identifying information for the selected exemplar genetic element corresponding to the matched genetic element.

In some embodiments, two or more matched genetic elements are provided that match to the same segment of the query nucleic acid sequence. In some embodiments, the query nucleic acid sequence is annotated with identifying information for two or more selected exemplar genetic elements corresponding to two or more matched genetic elements. In such instances, selection of the identifying information from among the two or more selected exemplar genetic elements corresponding to the two or more matched genetic elements may be required. For example a set of annotation rules may be applied in cases where the query nucleic acid sequence is capable of being annotated with identifying information for two or more selected exemplar genetic elements corresponding to two or more matched genetic elements.

In some embodiments, if the two or more matched genetic elements that match to the same segment of the query nucleic acid sequence are of a different type (as indicated in the type field corresponding to each of the exemplar genetic elements, e.g., gene, genetic region, insertion sequence, inverted repeat, direct repeat, etc.), the identifying information for two or more selected exemplar genetic elements corresponding to the two or more matched genetic elements is used to annotate the same segment of the query nucleic acid sequence.

In some embodiments, if the two or more matched genetic elements that match to the query nucleic acid sequence are non-overlapping, the identifying information for two or more selected exemplar genetic elements corresponding to the two or more matched genetic elements is used to annotate the query nucleic acid sequence. As used herein, the term “non-overlapping” refers generally to two annotations on the same query nucleic acid sequence but positioned such that they do not overlap. In a query nucleic acid sequence that includes non-overlapping segments, both annotations are made and are present on the annotated query nucleic acid sequence and there is no conflict. Two sequences may be non-overlapping if less than 100% of the sequences are identical, e.g., less than 95%, less than 90%, less than 85%, less than 80%, less than 75%, less than 70%, less than 70%, less than 65%, less than 60%, less than 55%, less than 50%, less than 45%, less than 40%, less than 35%, less than 30%, less than 25%, less than 20%, less than 15%, less than 10%, less than 5%, or the sequences are 0% identical.

In some embodiments, if the two or more matched genetic elements that match to the same query nucleic acid sequence are overlapping, a choice between the identifying information for two or more selected exemplar genetic elements corresponding to the two or more matched genetic elements must be made, or whether or not both identifying information need to be kept on the annotated query nucleic acid sequence. As used herein, the term “overlapping” refers to two different exemplar genetic elements that match the same start and end positions on the query nucleic acid sequence. In some embodiments, the two or more matched genetic elements that match to the same segment of the query nucleic acid sequence may be partially overlapping. Partially overlapping sequences are treated as if they do not overlap at all.

In some embodiments, if the two or more matched genetic elements that match to the same segment of the query nucleic acid sequence have different calculated matching algorithm scores, identifying information for the selected exemplar genetic element corresponding to the matched genetic element with the highest calculated matching algorithm score is used to annotate the segment of the query nucleic acid sequence.

In some embodiments, if the two or more matched genetic elements that match to the same segment of the query nucleic acid sequence have identifying information (e.g., the first three or six letters of the identifying information for the two or more matched genetic elements are identical), then the matched genetic element with the longer identifying information is used to annotate the segment of the query nucleic acid sequence.

In some embodiments, if the two or more matched genetic elements that match to the same segment of the query nucleic acid sequence have the same identifying information and the same calculated matching algorithm scores, then the matched genetic element with the lower value as indicated in the identification field of the relational database is used to annotate the segment of the query nucleic acid sequence.

In some embodiments, three or more matched genetic elements are provided that match to the same segment of the query nucleic acid sequence. In such instances, selection from among the identifying information for the three or more selected exemplar genetic elements corresponding to the three or more matched genetic elements may be required. For example a set of annotation rules may be applied in cases where the query nucleic acid sequence is capable of being annotated with identifying information for three or more selected exemplar genetic elements corresponding to three or more matched genetic elements. In some embodiments, if three or more matched genetic elements match to the same segment of the query nucleic acid sequence, then the set of annotation rules may be repeated until all conflicts have been resolved for the segment of the query nucleic acid sequence that is to be annotated.

As can be appreciated by those of skill in the art, any annotation rules or any combination of annotation rules may be implemented together with the methods as described above. Persons of skill in the art will be able to determine which combination of annotation rules best suit their needs, and accordingly, will be able to implement such rules for use together with the methods described above.

In some embodiments, the set of annotation rules is repeated for every segment of the query nucleic acid sequence in which a conflict arises. In some embodiments, after resolution of each and every conflict, a query nucleic acid sequence may be fully annotated. In some embodiments, after resolution of each and every conflict, a query nucleic acid sequence may be fully annotated, but may include one or more gap sequences that are not annotated.

As used herein, the term “gap sequence” refers to any nucleic acid sequence or segment thereof that is not annotated during a first round of the annotation process. A gap sequence may be located at a terminal end of the query nucleic acid sequence, or may be located within the query nucleic acid sequence flanked on either side with annotated sequences.

In some embodiments, a gap sequence within a query nucleic acid sequence may be annotated by matching the gap sequence to the exemplar nucleic acid sequence for one or more of the exemplar genetic elements in a relational database, wherein the matching includes applying a corresponding matching algorithm identified in the identifier for a matching algorithm field for the exemplar genetic element to compare the gap sequence with the exemplar nucleic acid sequence for the exemplar genetic element, similar to the methods described above for annotating a query nucleic acid sequence.

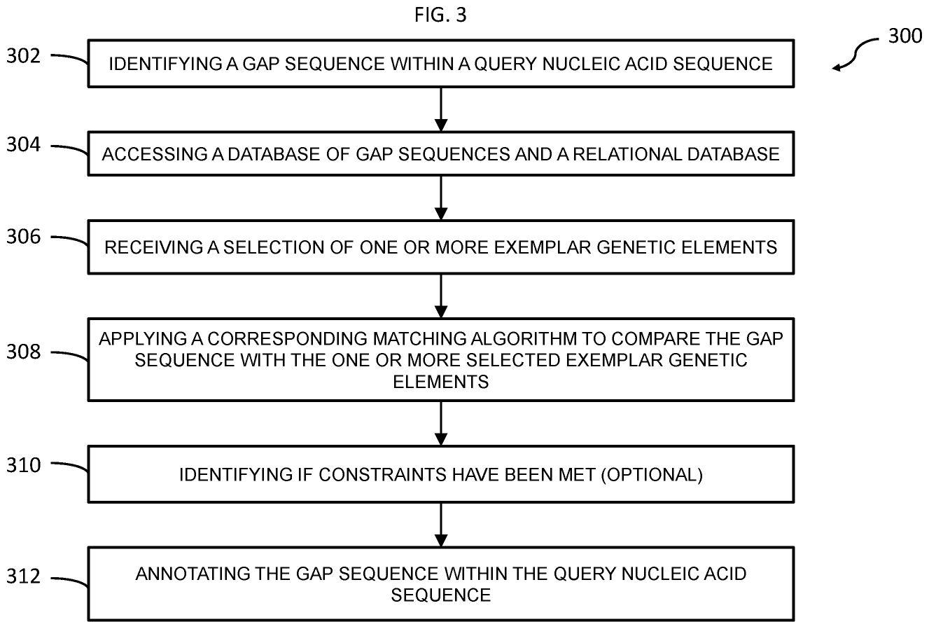

In some embodiments, the annotation process as described above may not detect occurrences of exemplar genetic elements on the query nucleic acid sequence if, for example, only a portion of the exemplar genetic element is present in the query nucleic acid sequence, even if the portion of the exemplar genetic element present in the query nucleic acid sequence is identical to a portion of the exemplar genetic element of the relational database. In such cases, the portion of the exemplar genetic element present in the query nucleic acid sequence, even if it is identical to the exemplar genetic element of the relational database, may not be matched with the query nucleic acid sequence if, for example, it is of a shorter length that fails to meet the minimum identity match criterion that corresponds with the exemplar genetic element. In such embodiments, the unmatched sequences of the query nucleic acid sequence may be presented as a gap sequence within the query nucleic acid sequence. To avoid issues arising from these embodiments, and without losing accuracy of the annotation process, a database of the gap sequences may be created, and the annotation process above may be repeated using the gap sequences within the query nucleic acid sequence and matching each of the gap sequences to the exemplar nucleic acid sequence for one or more of the exemplar genetic elements in a relational database. In such embodiments, the same matching algorithm and constraints corresponding to each of the one or more exemplar genetic elements may be maintained. For example, FIG. 3 is a flow diagram of a method 300 for annotating a gap sequence within a query nucleic acid sequence, according to an example embodiment. In step 302, a first annotation process may identify a gap sequence within the query nucleic acid sequence. Step 304 includes accessing a database of gap sequences, e.g., a relational database, and accessing a relational database including exemplar genetic elements as described herein. Step 306 includes receiving a selection of one or more exemplar genetic elements from the relational database including exemplar genetic elements. It should be noted that step 306 may occur before, after, or simultaneously with step 304. In step 308, a corresponding matching algorithm is applied to compare the query nucleic acid sequence (here a gap sequence) with the one or more selected exemplar genetic elements. A minimum identity match criterion may be applied in a similar manner to that described for a first round of the annotation process. Step 310 includes identifying if constraints, if any, have been met, e.g., in a manner similar to that described for a first round of the annotation process. In step 312, the gap sequence within the query nucleic acid sequence is annotated with identifying information of any matched genetic element, e.g., where the results of the matching algorithm meet the minimum identity match criterion corresponding to the selected exemplar genetic element.

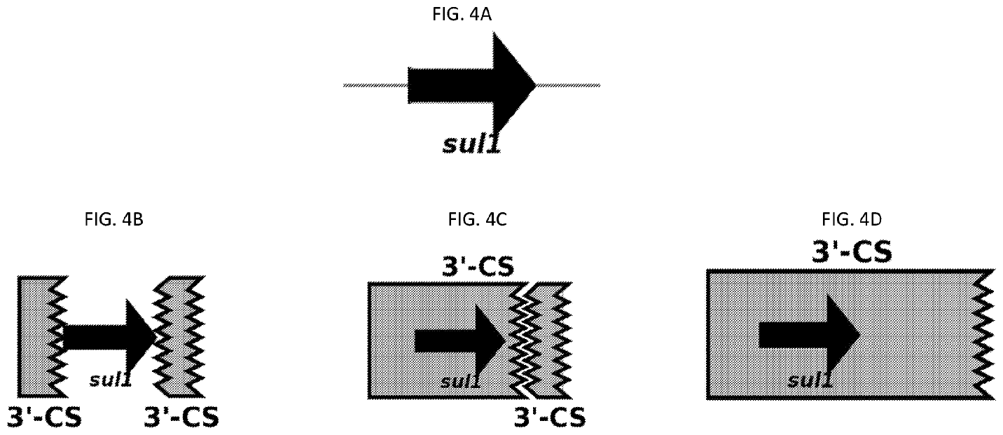

In some embodiments, since the annotation process described above may yield both the position of the match within the query nucleic acid sequence as well as the position of the match to an exemplar genetic element of the database, the matched element may be mapped back to its location within the query nucleic acid sequence and used to determine which nucleotides of the matched exemplar genetic element are missing from the query nucleic acid sequence. For example, FIGS. 4A-D show the different type of gap sequences that may be identified within a query nucleic acid sequence. FIG. 4A depicts, for example, sul1 flanked by gap sequences (horizontal lines) which may be annotated by the above described method.

In some embodiments, a gap sequence is a truncated sequence of an exemplar genetic element. In some embodiments, a truncated sequence of an exemplar genetic element that is present within the query nucleic acid sequence may overlap with a complete exemplar genetic element present within the query nucleic acid sequence. For example, FIG. 4B shows a complete gene within a truncated sequence of an exemplar genetic element within a query nucleic acid sequence. As such, the truncated sequence of the exemplar genetic element may not be fully included in gap sequences and thus, the overlapping portion of the truncated sequence of the exemplar genetic element may not be annotated. In some embodiments, each truncated end of the truncated sequence of an exemplar genetic element is tested to see if the nucleotide adjacent to the truncated end, even if that nucleotide is already annotated by a different exemplar genetic element, can be annotated. In other words, each truncated end of the truncated sequence of an exemplar genetic element is expanded. For example, FIG. 4C shows the expansion of the truncated sequence to the left of sul1. This process may be referred to as gap expansion.

In some embodiments, to ensure that the gap expansion process is accurate and allows for minor differences between the exemplar nucleic acid sequence of the exemplar genetic element in the relational database compared to the query nucleic acid sequence, the missing ends of truncated sequences are compared with the nucleotide sequence of adjacent annotations within the query nucleic acid sequence. In some cases, if the missing ends of truncated sequences match with the nucleotide sequence of adjacent annotations within the query nucleic acid sequence, but the identifying information is different, then the truncated sequence is expanded and the identifying information for both sequences are kept so that they overlap. In some cases, if the missing ends of truncated sequences match with the nucleotide sequence of adjacent annotations within the query nucleic acid sequence, and the identifying information are the same, then the matched sequences are merged into a longer matched genetic element.

In some embodiments, gap expansion is repeated until the truncated end of the truncated sequences reaches the completed end of the adjacent exemplar nucleotide sequence of the adjacent exemplar genetic element. In some embodiments, gap expansion is repeated until the end of the query nucleic acid sequence is reached. In some embodiments, gap expansion is repeated until there is no longer any missing nucleotide of the truncated sequence of an exemplar genetic element (FIG. 4D). In some embodiments, gap expansion is repeated until the query nucleic acid sequence does not match the missing nucleotide of the truncated sequence of gap being expanded.

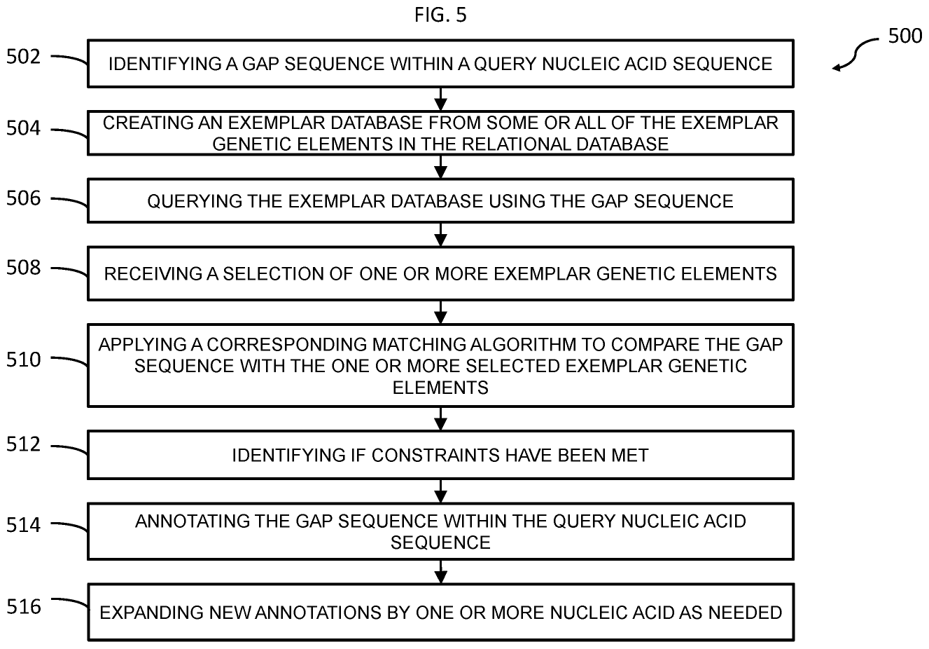

Accordingly, a computer-implemented method for annotating a query nucleic acid sequence according to the present disclosure may further include: expanding an end of a truncated sequence by one or more nucleotides to provide an expanded truncated sequence; and annotating the expanded truncated sequence by matching the expanded truncated sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the expanded truncated sequence with the exemplar nucleic acid sequence for the exemplar genetic element. FIG. 5 is a flow diagram of a method 500 for annotating a gap sequence within a query nucleic acid sequence, according to an example embodiment. In step 502, a first annotation process may identify a gap sequence within the query nucleic acid sequence. An exemplar database from some or all of the exemplar genetic elements within the relational database may be created 504. Step 506 includes accessing the exemplar database, e.g., a relational database, using the gap sequence. Step 508 includes receiving a selection of one or more exemplar genetic elements from the relational database including exemplar genetic elements. It should be noted that step 508 may occur before, after, or simultaneously with step 506. In step 510, a corresponding matching algorithm is applied to compare the query nucleic acid sequence (here a modified gap sequence) with the one or more selected exemplar genetic elements. A minimum identity match criterion may be applied in a similar manner to that described for a first round of the annotation process. Step 512 includes identifying if constraints, if any, have been met, e.g., in a manner similar to that described for a first round of the annotation process. In step 514, the gap sequence within the query nucleic acid sequence is annotated with identifying information of any matched genetic element, e.g., where the results of the matching algorithm meet the minimum identity match criterion corresponding to the selected exemplar genetic element. As needed, step 516 includes expanding new annotations by one or more nucleotides in one or both directions

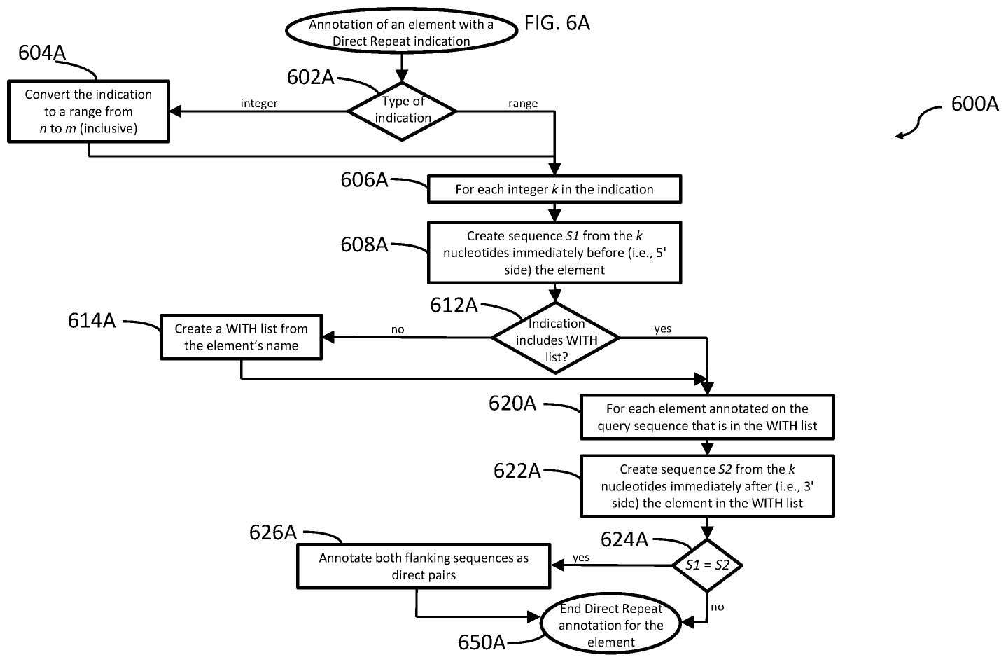

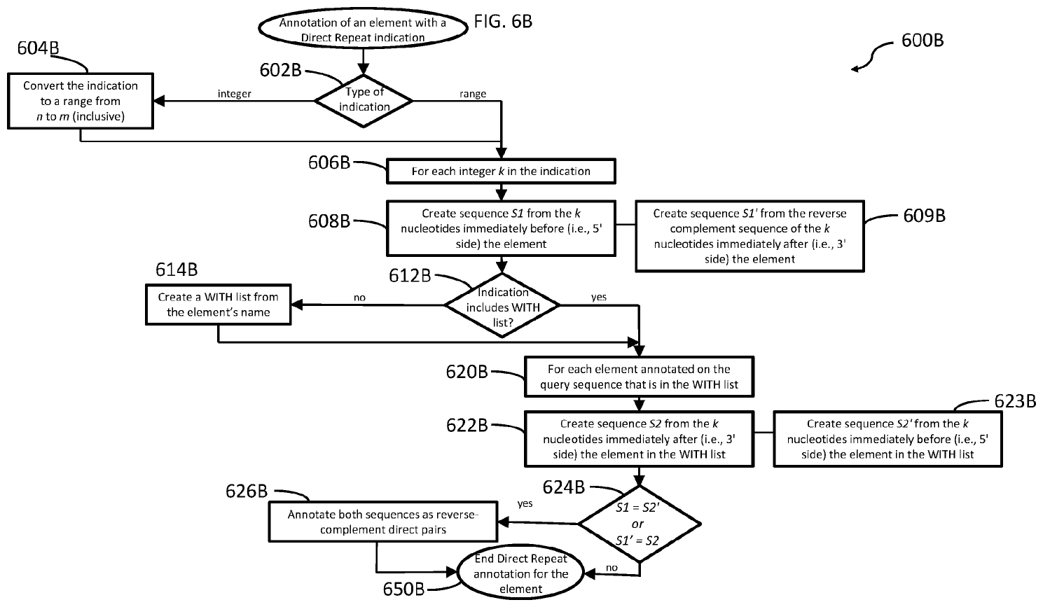

In some embodiments, a query nucleic acid sequence may include direct repeats to be annotated. In such cases, exemplar genetic elements of the relational database may be identified in the database as potentially associated with direct repeats. Sequences which flank sequences of the query nucleic acid sequence that match (as described herein) to the exemplar genetic elements are then checked for direct repeats. In one example embodiment, annotation of one element with a direct repeat indication within a query nucleic acid sequence can be done according to a method 600A shown in FIG. 6A. Depending on the value indicated in the direct repeats field (e.g., type of indication 602A), an integer may be converted to a range from n to m (inclusive) 604A. Once a range has been obtained for the direct repeat indication, for each integer k in the indication 606A, sequence S1 is created for the k nucleotides immediately before the element from the 5′ side 608A. If the indication does not include a “WITH” clause 612A then one is created with only the exemplar's name in it 614A. Every annotation on the same sequence that has a name that is included in the “WITH” clause, is checked for direct repeats in any of the combinations shown in FIG. 2A 620A. A sequence S2 is created for the k elements immediately after each element in the WITH list (i.e. on the 3′ side) 622A. If the sequences S1 and S2 are the same 624A, both flanking sequences are annotated as direct repeat pairs 626A. The direct repeat annotation process for the element is ended when there are no other annotations with names appearing in the “WITH” cause that have not been checked for direct repeats 650A.

In some embodiments, two matching annotated elements in the query sequence, are in opposite orientations relative to their exemplars in the relational database, and each of the two annotated elements has at least one end of the respective 3′ and 5′ ends in the respective exemplars, then the sequences immediately before or immediately after the respective 3′ and 5′ ends are checked for direct repeats that are reverse complements of each other, as shown in FIG. 2B. Reverse-Complement Direct Repeats are annotated according to the range of lengths specified in the relational database. In one example embodiment, reverse-complement direct repeats are annotated according to a method 600B shown in FIG. 6B. Depending on the value indicated in the direct repeats field (e.g., type of indication 602B), an integer may be converted to a range from n to m (inclusive) 604B. Once a range has been obtained for the direct repeat indication, for each integer k in the indication 606B, sequence S1 is created for the k nucleotides immediately before the element from the 5′ side 608B and a second sequence S1′ is created for the reverse complement sequence of the k nucleotides immediately after the element 609B. If the indication does not include a “WITH” clause 612B then one is created with only the exemplar's name in it 614B. Every annotation on the same sequence that has a name that is included in the “WITH” clause, is checked for direct repeats in any of the combinations shown in FIG. 2B 620B. A sequence S2 is created for the k elements immediately after each element in the WITH list (i.e. on the 3′ side) 622B. A sequence S2′ is created for the k elements immediately before each element in the WITH list (i.e. on the 5′ side) 623B. If S1 matches S2′ or if S1′ matches S2 624B, then the matching pair are annotated as reverse complement direct repeats 626B. The direct repeat annotation process for the element is ended when there are no other annotations with names appearing in the “WITH” cause that have not been checked for direct repeats 650B.

Assembly:

Using the methods for annotating a query nucleic acid sequence as described herein, larger assemblies of annotations may be generated according to observed patterns. In some embodiments, subject computer-implemented methods for annotating a query nucleic acid sequence further include annotating an assembly of annotations made to the query nucleic acid sequence. In such embodiments, the process of annotating the assembly of annotations includes: arranging a sequence for a first matched genetic element and a sequence for a second matched genetic element into a series of sequences for matched genetic elements; and processing the series of sequences for matched genetic elements using a parsing algorithm according to a predetermined set of parsing rules. In some embodiments, the sequences for a first and second matched genetic element are arranged by their starting position on the query nucleic acid sequence (e.g., their 5′ position). In some embodiments, the sequence for a first matched genetic element may be completely overlapping a second matched genetic element (e.g., a first smaller matched genetic element completely within a larger second matched genetic element), and the smaller matched genetic element's annotation may be attached to the larger matched genetic element, and the smaller matched genetic element removed from the assembly. In other words, in embodiments wherein when the sequence for the first matched genetic element is completely overlapped by the second for the second matched genetic element, the annotation for the first matched genetic element may be removed from the assembly.

In some embodiments, the process of annotating an assembly of annotations includes processing the series of matched genetic elements using any parsing algorithm and according to a predetermined set of parsing rules. Suitable parsing algorithms and parsing rules are described in Tsafnat, G. et al., Bioinformatics (2011) 27(6):791-796, which is incorporated by reference in its entirety herein. In some embodiments, the parsing algorithm may encounter errors when annotating an assembly of annotations, and the parsing algorithm may be reset to continue the process of annotating the assembly of annotations from the position in which the error occurred. Any suitable parsing algorithm will be apparent to those of skill in the art for use in a process for annotating an assembly of annotations according to any of the methods set forth herein.

In some embodiments, annotating an assembly of annotations using a parsing algorithm results in a parse tree. As used herein, the term “parse tree” refers to a tree structure in which smaller matched genetic elements that form a pattern are attached to a larger matched genetic element that represents the pattern. In some embodiments, to convey the pattern as a readable text, any number of tree visualization methods may be used, e.g. indenting lower levels appearing under higher levels. In some embodiments, the pattern may be conveyed as machine-readable text using any suitable markup language available in the art. For example, a suitable markup language may be eXtensible Markup Language (XML), JavaScript Object Notation (JSON), and the like.

In some embodiments, using the machine readable representation of the assembly of annotations, a graphical representation can be generated. In the graphical representation, various symbols may be used to represent different annotated elements (e.g., types of annotated elements). For example, symbols that may be used to represent different annotated element types include: an arrow (e.g., an arrow pointing from the 5′ to 3′ direction) representing a gene, a solid lollipop representing a direct repeat, an open lollipop representing a reverse complement direct repeat, a line representing a short gap sequence, a dashed line representing a long gap sequence, a flag representing an inverted repeat, a pentagon representing an insertion sequence, a rectangle representing all other exemplar genetic element types. In some embodiments, various colors may be used to represent different meanings. For example, commonly annotated and important exemplar genetic elements may have fixed colors including, but not limited to: 3′-consensus sequences and 5′-consensus sequences in orange, gene cassettes in light blue, insertion sequences in white, introns in silver, genes in black, gaps in red, Tn5393 in purple. The use of various color palettes may be useful in distinguishing between annotated elements that occur multiple times, e.g., direct repeat pairs may share the same color.

In some embodiments, generating a graphical representation of the assembly of annotation may include the following steps: reading the XML; determining the depth for each annotated element by annotated element type and its depth in the parse tree; adjusting the length of the annotated elements; recalculating the position of each annotated element so that each annotated element are adjacent to each other as needed; determining the label containing identifying information for each annotated element and the position of the label; drawing the annotated elements using Scalable Vector Graphics (SVG) from the deepest annotated element to the shallowest annotated element; rendering the SVG to produce a bitmap; and encoding the SVG or bitmap as needed. In some cases, the step of determining the depth for each annotated element may follow a general organizational structure, e.g., annotated elements such as inverted repeats and direct repeats may always be presented at the highest depth; annotated elements such as genes should be presented deeper than the regions that contain them; and annotated elements such as gap sequences should be presented at the shallowest level so that all other annotated elements overwrite them. In some embodiments, the step of adjusting the length of the annotated elements occurs if the symbol used to represent an annotated element is wider than the length of the annotated element would otherwise scale to, or if the annotated element is shortened (e.g., when representing a long gap sequence). In some embodiments, the graphical representation may be displayed on a client device (e.g., computer monitor, smart phone screen, etc.).

Methods of Monitoring

The present disclosure provides computer-implemented methods for monitoring the genetic material within a defined physical location. Genetic material within a defined physical location may be obtained from a variety of sources. Such methods may find use in a variety of applications, for example, monitoring the spread of an epidemic, monitoring the prevalence of antibiotic resistance, provide guidance in making clinical decisions, and others.

In some embodiments, methods of annotating a query nucleic acid sequence as described herein are implemented together with the collection of samples containing the query nucleic acid sequence at various time points and locations. For example, a method of monitoring the genetic material of a population of organisms in a defined physical location may include: collecting a representative sample of the population of organisms from the defined physical location at one or more time points; obtaining nucleic acid sequences from each of the representative samples; annotating the nucleic acid sequences according to the subject annotation methods; and calculating a frequency of occurrence of a genetic element of interest in the population of organisms based on the annotation. Such methods of monitoring the genetic material of a population of organisms may provide information on, e.g., whether a genetic element of interest is present within the defined physical location, the frequency of occurrence of a genetic element of interest in a population of organisms in the defined physical location, or a change in the frequency of occurrence of a genetic element of interest over time in a population of organisms in the defined physical location.

A representative sample may be obtained from a person in the defined space by various methods known in the art, for example, by collecting a bodily fluid such as blood or mucus. In some embodiments the person is a patient in a hospital bed. In other embodiments the person is a clinician in a hospital ward. In other embodiments the person is any other person in the defined space.

In some embodiments, a representative sample may be obtained from a defined physical location by various methods known in the art, for example, by swabbing a surface of the defined physical location.

In addition, nucleic acid sequences may be obtained from representative samples by any method known to those of skill in the art, including purifying and/or amplifying the nucleic acid sequences and sequencing them on commercially available sequencing platforms.

In some embodiments, the representative samples are collected from a defined physical location at one or more time points, e.g., two or more, three or more, four or more, five or more, six or more, seven or more, eight or more, nine or more, ten or more, fifteen or more, twenty or more, thirty or more, forty or more, or fifty or more time points. The frequency of representative samples collected will depend on the type of monitoring to be performed. In some embodiments, the one or more representative samples are collected over a period of one or more days, one or more weeks, one or more months, one or more years, etc. In some embodiments, the one or more representative samples are collected from the defined physical location every ten minutes, every thirty minutes, every hour, every two hours, every day, etc. In some embodiments, the one or more representative samples are collected at a specific time during the day, e.g., 8:00 in the morning, 12:00 noon, 6:00 in the evening, and may depend on how busy the defined physical location is, in terms of foot traffic, budget, or how feasible the collection of a representative sample is.

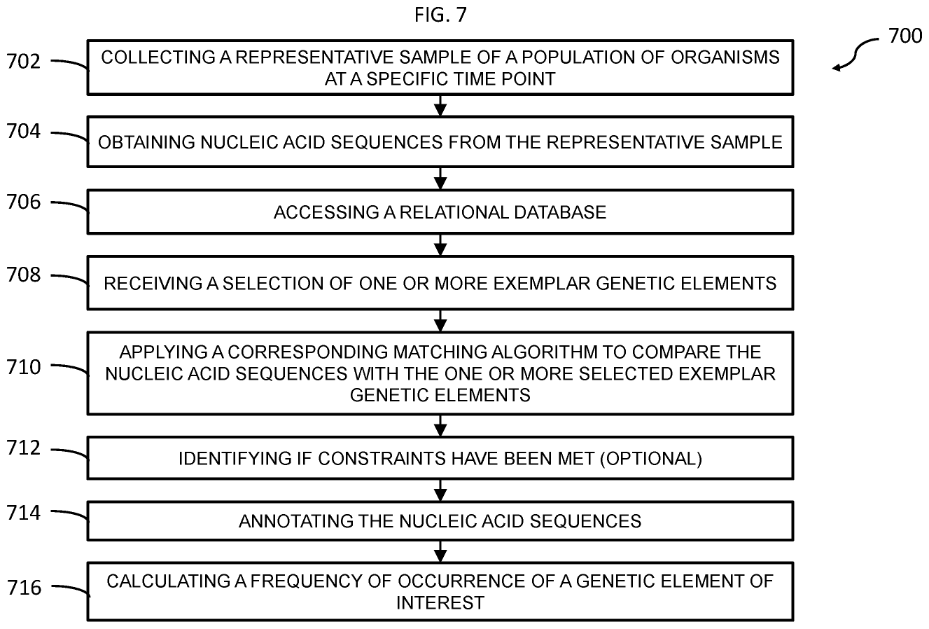

Accordingly, a method of monitoring the genetic material of a population of organisms in a defined physical location includes: collecting a representative sample of the population of organisms from the defined physical location at one or more time points; obtaining nucleic acid sequences from each of the representative samples; annotating the nucleic acid sequences by matching the nucleic acid sequences against a plurality of genetic elements in a relational database (e.g., as described herein); and calculating a frequency of occurrence of a genetic element of interest in the population of organisms based on the annotation. For example, FIG. 7 shows a flow diagram of a method 700 of monitoring the genetic material of a population of organisms in a defined physical location, according to an example embodiment. A representative sample of a population of organisms is collected at a specific time point 702 and nucleic acid sequences are obtained from the representative sample 704. The nucleic acid sequences (or portions thereof) may then be used as query nucleic acid sequences and annotated as described herein. For example, Step 706 includes accessing a relational database including a plurality of exemplar genetic elements as described herein. Step 708 includes receiving a selection of one or more of the exemplar genetic elements from the relational database. It should be noted that step 708 may occur before, after, or simultaneously with step 706. In step 710, a corresponding matching algorithm is applied to compare the query nucleic acid sequence with the one or more selected exemplar genetic elements. Step 712 includes identifying if constraints, if any, have been met. In step 714, the nucleic acid sequences are annotated with identifying information of any matched genetic element, e.g., as described elsewhere herein. In step 716, the frequency of occurrence of a genetic element of interest (e.g., antibiotic resistance gene) may be calculated.

As used herein, the term “frequency of occurrence” refers to, for example, the number of times a genetic element of interest is used to annotate query nucleic acid sequences obtained from a particular sample obtained from a defined physical location. For example, the frequency of occurrence of a genetic element of interest may refer to the number of times the genetic element of interest is used to annotate query nucleic acid sequences obtained from a particular sample obtained from a defined physical location at a given time point.

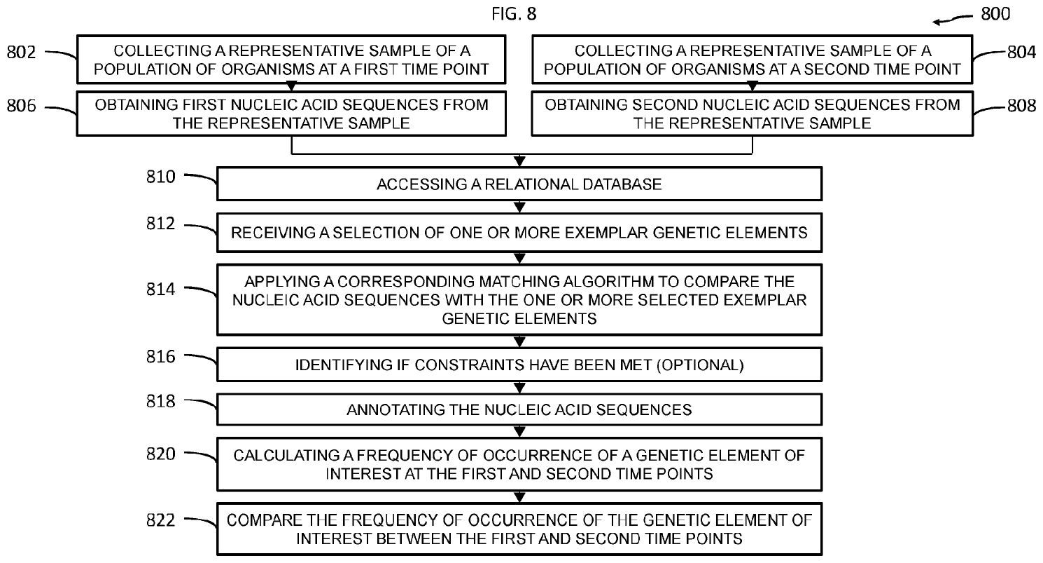

In one embodiment, the method of monitoring the genetic material of a population of organisms in a defined physical location includes collecting a representative sample of the population of organisms from the defined physical location at two or more time points; and comparing the frequency of occurrence of the genetic element of interest at a first time point to the frequency of occurrence of the genetic element of interest at a second, later time point. For example, FIG. 8 shows a flow diagram of a method 800 of monitoring the genetic material of a population of organisms in a defined physical location, according to an example embodiment. A representative sample of a population of organisms is collected at a first and second time point 802, 804 and nucleic acid sequences are obtained from each of the representative samples 806, 808, to be used as query nucleic acid sequences in a computer-implemented method. Step 810 includes accessing a relational database, wherein the relational database includes a plurality of exemplar genetic elements and fields as described elsewhere herein. Step 812 includes receiving a selection of one or more exemplar genetic elements contained within the relational database. It should be noted that step 812 can be performed before, after, or simultaneously with step 810. In step 814, a corresponding matching algorithm is applied to compare the query nucleic acid sequences with the one or more selected exemplar genetic elements. Step 816 includes identifying if constraints, if any, have been met. In step 818, the query nucleic acid sequences are annotated with identifying information of any matched genetic element, which either meets the constraints corresponding to the selected exemplar genetic element or for which constraints are not present. In step 820, the frequency of occurrence of a genetic element of interest (e.g., antibiotic resistance gene) may be calculated for each of the time points, and compared 822. In some embodiments, the method further includes a step of generating a report showing the frequency of occurrence of the antibiotic resistance gene or a graphical representation thereof. In some such embodiments, the report shows a trend in frequency of occurrence of the antibiotic resistance gene over time.

In some embodiments, the frequency of occurrence of the genetic element of interest at a first time point is different compared to the frequency of occurrence of the genetic element of interest at a second, later time point. For example, when the genetic element of interest is an antibiotic resistance gene, an increase in the frequency of occurrence of the antibiotic resistance gene at the second time point relative to the first time point may indicate that the population of organisms in the defined physical location is exhibiting an increase in antibiotic resistance. Whereas a decrease in the frequency of occurrence of the antibiotic resistance gene at the second time point relative to the first time point may indicate that the population of organisms in the defined physical location is exhibiting a decrease in antibiotic resistance. In such embodiments, a value may be set for an alert identifier field corresponding to the genetic element of interest to raise an alert when a genetic element of interest is used to annotate a nucleic acid sequence, or when the frequency of occurrence of a genetic element of interest changes.

Utility

The present disclosure provides computer-implemented methods for annotating a query nucleic acid sequence include accessing a relational database that includes a plurality of exemplar genetic elements. Subject methods may find use in a variety of applications.

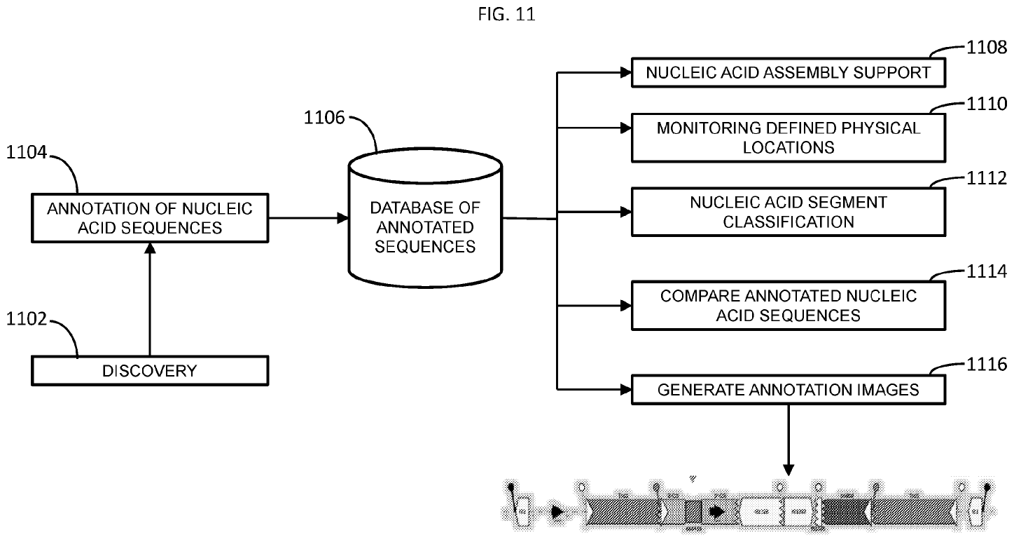

Referring to FIG. 11, FIG. 11 shows a flow diagram for several applications of the subject methods for annotating query nucleic acid sequences. Upon discovery 1102 of nucleic acid sequences (e.g., isolation and sequencing of query nucleic acid sequences), the nucleic acid sequences are annotated 1104 (e.g., according to one or more of the methods described herein) and may be stored in a database of annotated sequences 1106. Annotated nucleic acid sequences may find use in nucleic acid assembly support 1108, monitoring defined physical locations 1110, nucleic acid segment classification 1112, comparing annotated nucleic acid sequences 1114, generating annotation images 1116, and the like.

In some embodiments, subject methods may lead to discovery 1102. For example, subject methods may be used to discover mobile elements within a query nucleic acid sequence. For example, using the parsing algorithm and predetermined set of parsing rules as described elsewhere herein, it may be possible to craft specific rules that facilitate the identification of mobile elements based on surrounding exemplar genetic elements. In some embodiments, a potential mobile element may be identified as a region flanked by two ends of a mobile element. In some embodiments, the subject methods may be used to discover new gene cassettes associated with integrons, e.g., as described in Tsafnat, G., et al., BMC Bioinformatics (2009) 10:281, which is incorporated by reference herein in its entirety herein. In some embodiments, the subject methods may be used to discover novel gene cassettes that may confer antibiotic resistance, e.g., as described in Partridge, S. R. and Tsafnat, G., Antimicrob. Agents and Chemotherapy (2012) 56(8):4566-4567.

In some embodiments, subject methods may be used to facilitate and support nucleic acid assembly 1108, for example, in the assembly of nucleic acid strands from shorter sequences. Assembly of nucleic acid strands from shorter sequences is complicated by long repetitive regions that result from, e.g., auto-recombination, the presence of mobile genetic elements and other natural DNA events. In particular, when the repetitive regions are longer than the segments being assembled. In some cases, annotation of partially assembled sequences can reveal regions that are mobile and sites that could have recombined and indicate which regions are likely to have multiple copies indicating how assembly may continue.

The subject methods find particular use in the monitoring of defined physical locations 1110, for example, in the monitoring of pathogenic genes within a population of organisms within a defined physical location. For example, the presence of specific antibiotic resistance genes may provide valuable information on treatment options and/or strategy for people who developed infections within the monitored location or who were exposed to the monitored location.

In some embodiments, subject methods facilitate nucleic acid segment classification 1112, i.e., facilitate the accurate annotation of nucleic acid sequences. Accurate annotation of nucleic acid sequences using subject methods can be used to identify, e.g., chromosomes, plasmids, mobile elements, specific regions of DNA that uniquely identify a strain (e.g., a bacterial strain, a viral strain, etc.), virulence genes, specific gene variants of clinical significance, antibiotic resistance genes, etc. For example, accurate identification of sequences through annotation may facilitate distinguishing bacterial strains from one another through subtle changes in their DNA sequences. This may be important in applications including, e.g., infection identification and control, identifying pathogenic strains, identifying virulence and resistance risks, etc.

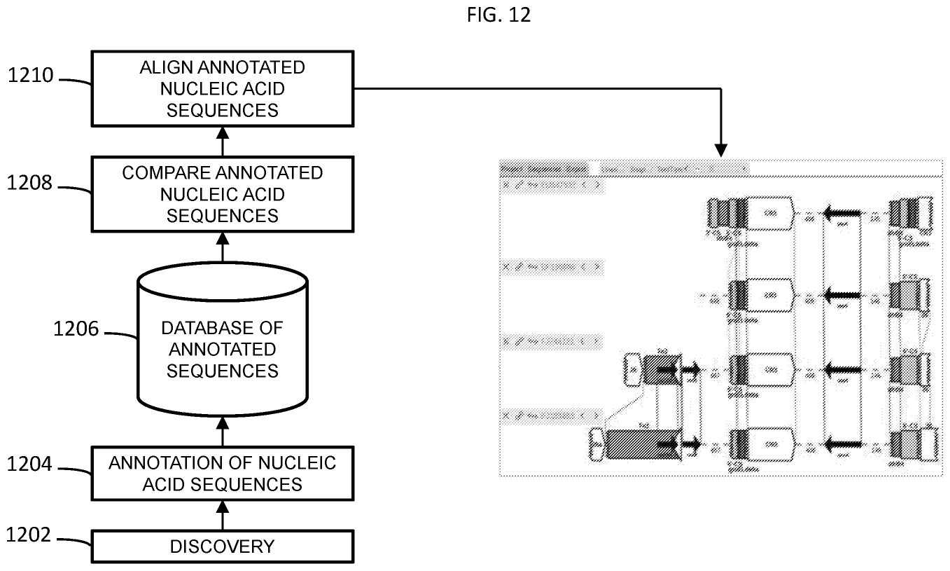

Subject methods may find use in the comparison of two or more nucleic acid sequences 1114. For example, discovering gene functions and evolution largely relies on comparing two or more nucleic acid strands, but is computationally difficult in part because of the large number of nucleotides involved. Effective comparison of two or more nucleic acid sequences may be facilitated by the use of subject methods described herein. In some embodiments, comparison of two or more nucleic acid sequences may include the following steps: using the subject methods described herein to annotate each nucleic acid sequence; representing each nucleic acid sequence by its annotated information; and comparing the order of annotation of each nucleic acid sequence in order to identify differences (e.g., transposition mutations, etc.). FIG. 12 shows a flow diagram for comparing and aligning annotated nucleic acid sequences. Upon discovery of nucleic acid sequences 1202 (e.g., isolating and sequencing of nucleic acid sequences), nucleic acid sequences are annotated 1204 and may be stored in a database of annotated sequences 1206. Annotated sequences may then be compared 1208 and aligned 1210, e.g. aligned according to the annotated segments of the nucleic acid sequences as shown in the sample screenshot. Once the nucleic acid sequences are aligned, differences may be identified.

In some embodiments, annotation images may be generated 1116 from nucleic acid sequences annotated by any of the subject methods. In such embodiments, the annotation images may facilitate the comparison of annotated nucleic acid sequences via the alignment of annotated segments within a nucleic acid sequence.

In some embodiments, subject methods may be used to discover new variants of a known gene. In such embodiments, several steps may be followed: setting a high minimum identity match criterion for all known variants of the known gene, or setting specific constraints to identity all known variants of the known gene; adding a new exemplar genetic element to the relational database with a similar nucleotide sequence to the nucleotide sequence of the known variants, wherein the new exemplar genetic element is set with a low minimum identity match and no constraints; and adding an alert value (e.g., in the alert field) for the new exemplar genetic element such that an alert is raised whenever the new exemplar genetic element is used in an annotation, indicating that a new variant of the known gene has been identified. In such embodiments, the new exemplar genetic element may be set with a low minimum identity match and no constraints such that: any of the known variants would be annotated as the new exemplar genetic element if the variants' exemplar genetic elements are excluded from the annotation; and any similar nucleotide sequence that failed the constraints of all the variants would still be annotated by the exemplar genetic element of the known gene.

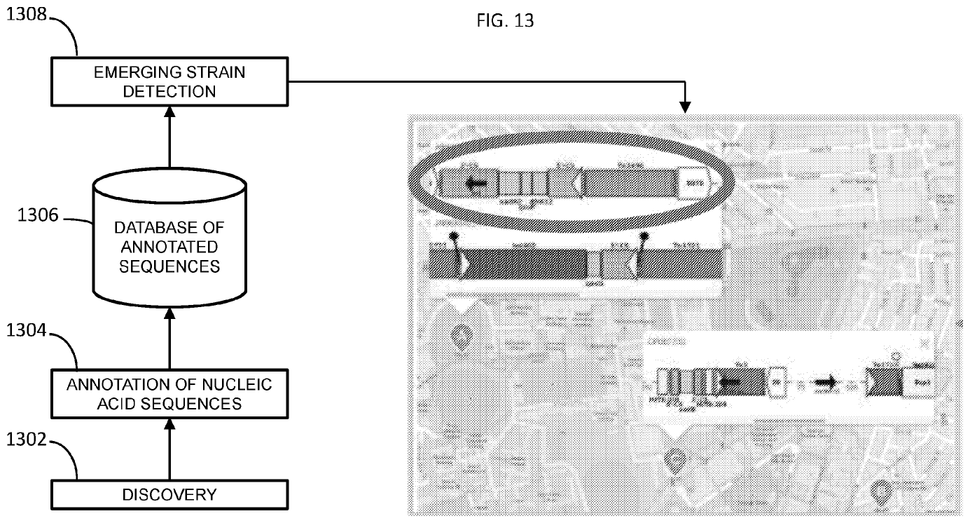

Referring to FIG. 13 which shows a flow diagram, in some embodiments, subject methods may be used to provide support in the early detection of emerging strains 1308, e.g., emerging microbial strains. Upon discovery of nucleic acid sequences 1302 (e.g., isolation and sequencing of a representative sample obtained from a defined physical location), nucleic acid sequences are annotated 1304 and may be stored in a database of annotated sequences 1306. Methods for annotating sequences as described herein may facilitate the detection of emerging strains 1308. For example, genetic monitoring for emerging microbial strains can provide early warning for potential new diseases and epidemics, and direct research on the new strains. Detecting a new strain is a distinct problem relevant to regular monitoring of a defined physical location because the new strain may include new genetic elements or new combinations of genetic elements that are unknown in the art. In some embodiments, to detect an emerging strain in a defined physical location, in addition to the subject methods described herein for monitoring a defined physical location, discovering new genes and gene variants from annotations, the following steps may be performed to discover emerging microbial strains: using historical data of all nucleic acid sequence annotations previously found in the same defined physical location, recording all annotations that have previously and/or recently been identified in the defined physical location; and whenever a new annotation is discovered within the defined physical location, comparing it with the historical annotations and alert a user (e.g. by email, text message, mobile application notification, etc.) or another device (e.g. by invoking a pre-set procedure) to report that a new annotation has been discovered. In some cases, detecting an emerging strain in a defined physical location further includes identifying and analyzing gap sequences in the annotation and repeating the annotation process with increased sensitivity (e.g., by modifying the minimum identity match for specific exemplar genetic elements); and using subject methods described herein for new gene variant discovery; and alerting a user (e.g. by email, text message, mobile application notification, etc.) or another device (e.g. by invoking a pre-set procedure) to report on new gene variants that have been identified. In one example, as depicted in FIG. 13, three defined physical locations A, B, and C are monitored for an emerging strain which is detected in defined physical location A indicated by the circled annotated sequence.

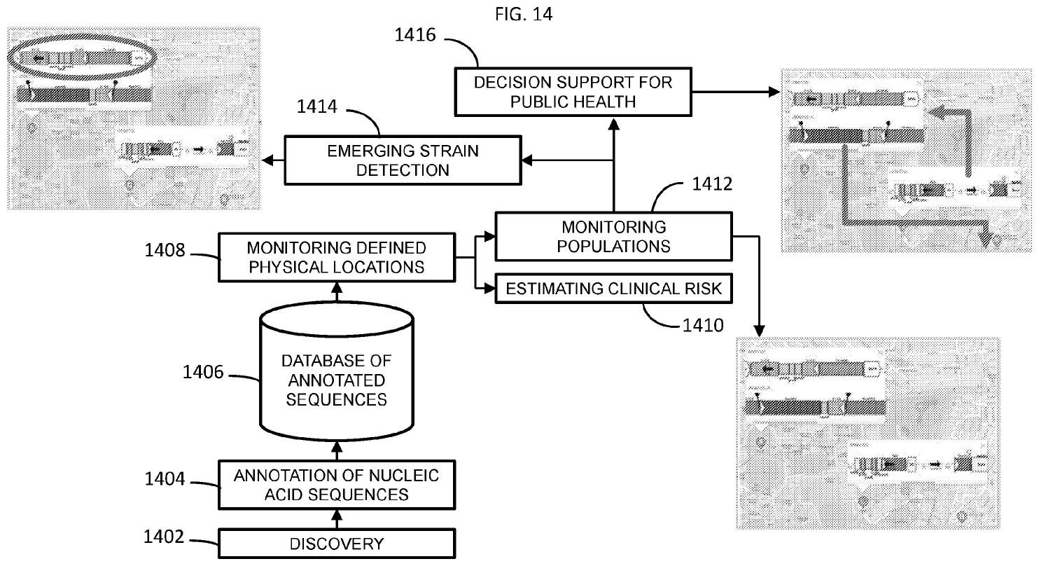

FIG. 14 provides a flow diagram for the use of subject methods in monitoring defined physical locations. Upon discovery of nucleic acid sequences 1402 (e.g., isolation and sequencing of a representative sample obtained from a defined physical location), nucleic acid sequences are annotated 1404 and may be stored in a database of annotated sequences 1406. The annotated sequences may be used in monitoring defined physical locations 1408, for example, in monitoring populations 1412 or in estimating clinical risk 1410. Monitoring populations 1412 may lead to the detection of an emerging strain 1414, and/or provide guidance in decision support for public health 1416.

In some embodiments, subject methods may be used for monitoring populations 1412, e.g., the spread of pathogenic genes within a population or environment. In some cases, the emergence of epidemics illustrates the mechanism by which pathogens spread. Genes follow similar and distinct patterns of spread. In some embodiments, subject methods can be used to monitor defined physical locations, and coordinated monitoring can provide a picture of the movement of genes, laying out the risks from each defined physical location to reveal a community structure (FIG. 14). The visualization may show how genes and organisms are spread geographically over time so that actions to control such spread may be identified. In such embodiments, monitoring an environment using subject methods may aid in estimating clinical risk 1410, e.g., provide predictions about properties of infections detected within the environment. In particular, clinically relevant properties such as pathogenicity, virulence and antibiotic resistance of certain identified genetic elements may be made. In some embodiments, using subject methods to monitor nucleic acid sequences within an environment may provide the frequency of occurrence of the nucleic acid sequences. In some embodiments, the combination of the data obtained from multiple defined physical locations can be used to make predictions on future trends of spread. In such cases, a class of algorithms called Machine Learning may be used to make a prediction from historically available data. In other cases, a Bayesian Network algorithm can be used to perform the following: model relationships between genetic elements in the environment, e.g., the distance between defined physical locations (e.g., beds in a hospital room); calculate the frequency of occurrence of pathogenicity, virulence and antibiotic resistance genes in each of the defined physical locations; and calculate a probability that an infected patient that came into contact with any or all of the monitored defined physical locations has an infection that carries any of the monitored genetic elements. Any form of predictive modelling known in the art may be used to predict the occurrence of genetic elements as described above, for example, parametric, non-parametric and semi-parametric regression models. In addition, predicting the occurrence of genetic elements as described above may be implemented with further advances in artificial intelligence.

In some embodiments, based on the genes predicted to be associated with an infection, clinical or other action may be taken before clinical samples are obtained from a patient to be pathologically assessed. For example, the administration of a certain antimicrobial drug may be avoided if a prediction that the infection is resistant to the drug is made. For example, a patient may be quarantined if the infection is predicted to be highly virulent. In some embodiments, using subject methods, in order to support predictions, the predictive information may be presented in the form of a paper or electronic chart that is displayed near the monitored defined physical location such that decision makers (e.g., doctors and nurses) can see any predicted environmental risk before making any decisions. For example, a hospital room may be monitored for the occurrence of antibiotic resistance genes and a prediction risk chart may be displayed at any suitable location in or near the hospital room, e.g., on the door to the hospital room, so that clinicians can review the chart before prescribing antibiotics to any patients within. In such cases, the prediction risk chart may be replaced every time predictions are updated and/or at regular intervals.

In some embodiments, based on the genes predicted to be associated with an infection, clinical or other action may be taken based on clinical samples obtained from a patient to be pathologically assessed. For example, the administration of a certain antimicrobial drug may be avoided if a prediction that the infection is resistant to the drug is made. For example, a patient may be quarantined if the infection is predicted to be highly virulent. In some embodiments, using subject methods, in order to support predictions, the predictive information may be presented in the form of a paper or electronic chart that is displayed near the patient such that decision makers (e.g., doctors and nurses) can see any predicted specific risk before making any decisions. In such cases, the predictive information may be replaced every time predictions are updated and/or at regular intervals.

In some embodiments, subject methods may be used to provide decision support for public health 1416. For example, using monitored information from several defined physical locations, such as different rooms in a hospital ward, health policy decisions may be made. For example, extra cleaning for the ward may be ordered. In other examples, hospital drug dispensaries may be adjusted to accommodate the future needs of clinicians (e.g., stocked with certain drugs that are predicted to overcome the occurrence of antibiotic resistance), contaminated equipment may be replaced, hand washing policies may be modified, prescription policies may be modified, and high-risk patients may be diverted away from a contaminated hospital ward. Similarly, at a population level, vaccination, medicine stockpiling and infection control programs can be initiated, adjusted or informed using predictions and other decision support methods as described herein.

In some embodiments, subject methods may be used for curating databases of composite exemplar genetic elements such as integrons. A database (e.g., database of annotated sequences) including one or more nucleic acid sequences annotated by the subject methods (e.g., annotated composite nucleic acid sequences) may be developed. In some embodiments, each annotated composite nucleic acid sequence may be represented by its identifying name, type and/or other identifying information; each exemplar genetic element used to annotate each of the annotated composite nucleic acid sequences is ordered according to their relative position in the annotated composite nucleic acid sequence; delimit the ordered elements by use of a delimiter character not used in the identifying information (such as a semicolon ‘;’); and store the resulting string in a database along with an identifier of the nucleic acid sequence (e.g., accession number). In some embodiments, the curated database may facilitate the comparison of annotated composite nucleic acid sequences to track sources of infections, research the evolution of microorganisms, research complex cellular functions, estimate the prevalence of the nucleic acid sequence, etc.

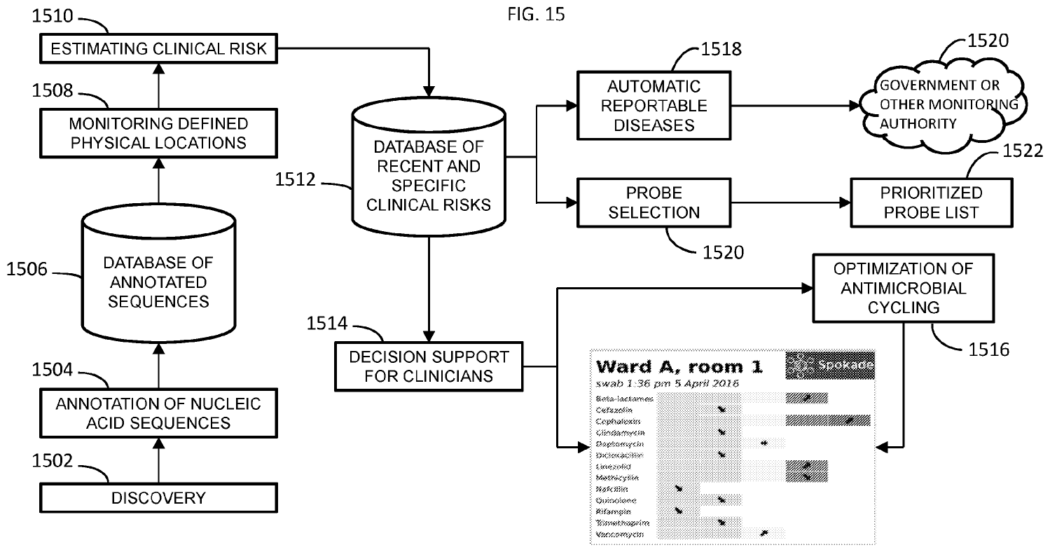

In some embodiments, subject methods may be used for the automatic and accurate reporting of reportable diseases and genes. For example, FIG. 15 provides a flow diagram showing how annotated sequences may be used for monitoring defined physical locations. Upon discovery of nucleic acid sequences 1502 (e.g., isolation and sequencing of a representative sample obtained from a defined physical location), nucleic acid sequences are annotated 1504 and may be stored in a database of annotated sequences 1506. The annotated sequences may be used to monitor defined physical locations 1508 and facilitate in the estimating of clinical risk 1510 for a given nucleic acid sequence (e.g., antibiotic resistance gene). Clinical risks associated with specific nucleic acid sequences may be stored in a database of recent and specific clinical risks 1512, which may be accessed to provide decision support for clinicians 1514. With access to a database of recent and specific clinical risks, a clinician may be able to optimize antimicrobial cycling 1516. For example, in the example screenshot of a resistance-risk chart for ward A room 1, a high risk of resistance to cephalexin is displayed. As such, using subject methods for monitoring a defined physical location, the development of resistance within the defined physical location may be predicted and clinicians may be able to inform their decisions on the type of drugs to administer and/or to avoid.

As part of a public health policy, health authorities may require healthcare providers to report diagnoses of certain communicable diseases. Using subject methods to monitor genetic material, it may be possible to report not only on disease diagnosis, but also on specific genes (e.g., antibiotic resistance genes) that can move independently of the diagnosed infection and that have clinical significance to public health. In such embodiments, the reportable exemplar genetic elements may be designated as such in the relational database using the alert field, with a description of an action to be performed. Monitoring of genetic material is performed as described herein. In such embodiments, whenever a reportable exemplar genetic element is used to annotate a query nucleic acid sequence using the subject methods, the action to be performed associated with that element will be performed automatically. For example, in FIG. 15, accessing a database of recent and specific clinical risks 1512 may provide a list of automatic reportable diseases 1518, which can be automatically sent to the government or other monitoring authority 1520 as part of a public health policy.

In some embodiments, accessing a database of recent and specific clinical risks 1512 may facilitate probe selection 1520 and provide a prioritized probe list 1522. Probes developed based on annotated sequences that may contribute to clinical risk may then be used for rapid testing of individuals.

### Systems and Devices

Exemplary systems and devices of the present disclosure are now described with reference to the Figures.

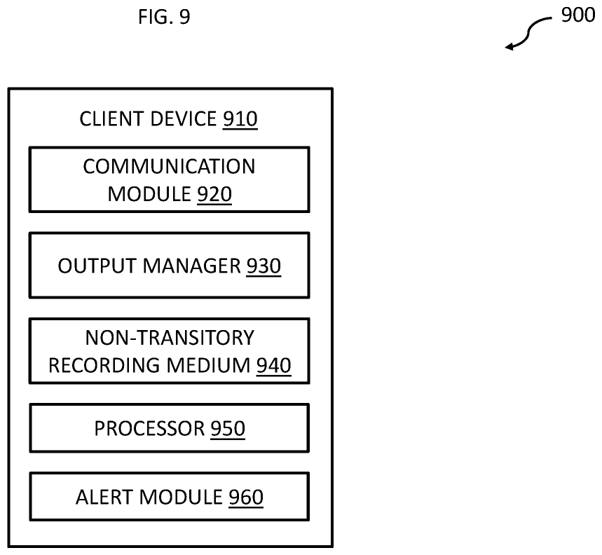

FIG. 9 illustrates a block diagram of a system for annotating a query nucleic acid sequence. As illustrated in FIG. 9, the system 900 generally includes a client device 910, a communication module 920, an output manager 930 for communicating output to a user and a non-transitory computer-readable recording medium 940 containing instructions, which when executed by one or more processors 950, cause the one or more processors to perform one or more steps of the subject methods for annotating the query nucleic acid sequence. In some embodiments, the non-transitory computer-readable recording medium 940 contains instructions, which when executed by one or more processors 950, cause the one or more processors to perform any of the methods described herein.

A system according to one embodiment optionally includes an alert module 960 for alerting the user when a specific genetic element has been annotated. In embodiments where the user is in a remote location, the alert module is configured to transmit the alert to the user, e.g., via electronic mail, a short message service, a mobile application notification, and the like.

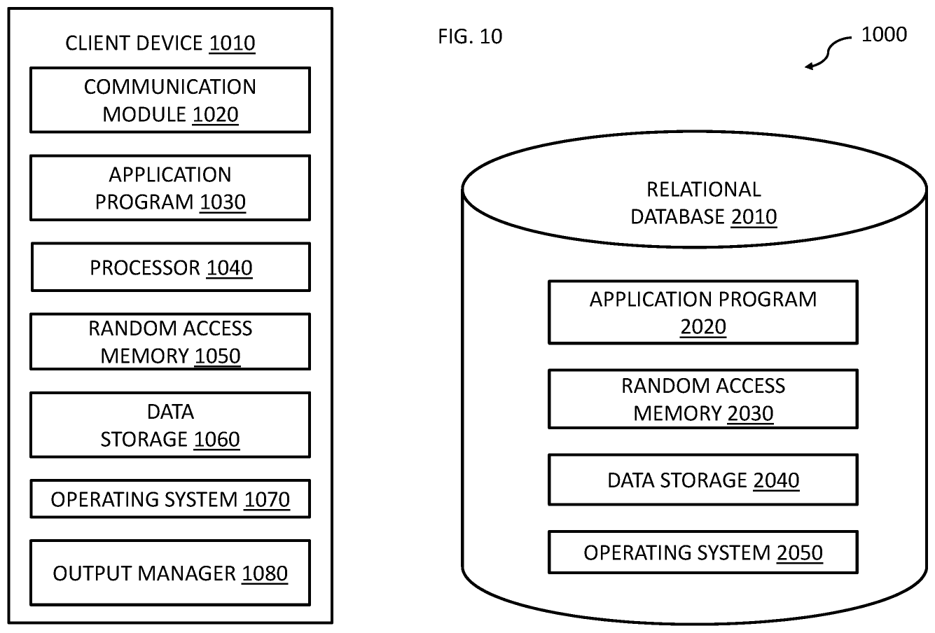

FIG. 10 illustrates a block diagram of a system for annotating a query nucleic acid sequence, according to one example embodiment. As illustrated in FIG. 10, the system 1000 generally includes a client device 1010, and a relational database 2010.

The client device 1010 may include, but is not limited to, a communication module 1020, an application program 1030 to execute commands or instructions to annotate the query nucleic acid sequence. The client device 1010 may further include a processor 1040, random access memory (RAM) 1050, permanent data storage 1060, an operating system 1070 and an output manager 1080. In other examples, the data storage may be either substituted with or supplemented by a cloud-based storage (not illustrated). In some embodiments, the query nucleic acid sequence may originate from the client device 1010, and the computer processor 1040 of client device 1010 may be programmed to transmit query nucleic acid sequence data to the relational database 2010. In some embodiments, the computer processor of the client device 1010 may be programmed to receive data from the relational database 2010, which may be displayed, for example, on the client device. The relational database 2010 may be housed in an independent unit, including, but not limited to, an application program 2020, a random access memory 2030, a data storage 2040, and an operating system 2050. In some embodiments, the computer processor of the client device may be programmed to transmit the query nucleic acid sequence data to a plurality of databases. In other examples, the client device may be programmed to transmit multiple query nucleic acid sequence data to a plurality of databases. The application program may be implemented by the operating system of the client device. In other examples, the application program 1030 may be stored in a non-transitory computer-readable recordable medium. In another example, the software application may be a web-based application and stored on an external server or external database (not illustrated).

A system according to such an embodiment optionally includes an alert module for alerting the user when a specific genetic element has been annotated. In embodiments where the user is in a remote location, the alert module is configured to transmit the alert to the user, e.g., via electronic mail, a short message service, a mobile application notification, and the like.

The methods, devices, and systems of the present disclosure can be used to improve technology, such as by improving the functioning of processes and machines (e.g., computers). In some cases, the methods, devices, and systems of the present disclosure can reduce the time (e.g., speed up the processing) for a computer to provide an answer, such as a sequence annotation or an analysis result. In some cases, the methods, devices, and systems of the present disclosure can reduce the memory requirements for a computer to provide an answer, such as a sequence annotation or an analysis result.

The methods, devices, and systems of the present disclosure can reduce the processing time of a given analysis by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more. The methods, devices, and systems of the present disclosure can reduce the memory requirements for a given analysis by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more.

The methods, devices, and systems of the present disclosure can be used to perform analyses not previously workable or solvable, or not workable or solvable without a computer system. For example, in some cases, the use of relational databases can enable analytic techniques which are not possible or not practical by other means.

Although the foregoing invention has been described in some detail by way of illustration and example for purposes of clarity of understanding, it should be readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### EXAMPLES

### Example 1: Anti-Microbial Resistance (AMR) Monitoring

A hospital is monitored for anti-microbial resistance. Environmental samples are taken periodically (e.g., daily) from different regions of the hospital (e.g., from each ward or unit). The environmental samples are sequenced and analyzed using methods of the present disclosure (e.g., using a matching algorithm to compare sample sequences to those in a relational database). The presence, absence, or abundance of traits (e.g., anti-microbial resistance (AMR)) are analyzed, tracked, and reported. A report is generated (see, e.g., FIG. 15) indicating levels of AMR risk and recent changes thereto. Hospital staff utilize the information in the report to make clinical decisions (e.g., rotating antibiotic usage, altering antibiotic dosages or treatment times).

A network of hospitals is similarly monitored. Results from these hospitals are aggregated, and monitoring of traits such as AMR is conducted across the network. Hospitals in the network are able to make clinical decisions utilizing information from their site and other relevant sites in the network.

### Example 2: Annotation

A query nucleic acid sequence was annotated. The query nucleic acid sequence was identified as belonging to CP011639 (Serratia marcescens). The annotation comprises the following tokens (i.e., annotations) in order as shown in Table 1. Numbers in parentheses indicate the region of the sequence with which the token is associated.

Gaps are designated here as nil-matches. The annotation process discovered some nil-matches to be new elements not in the original database. For example, the token 9.1.2.1.1 (from position 11029 to position 12284, inclusive, with length 1256 nucleotides) was predicted to be a mobile element such as an insertion sequence or transposon, due to its location within an interruption. Similarly, nil-matches located within cassette array structures could be identified as previously undocumented gene cassettes.

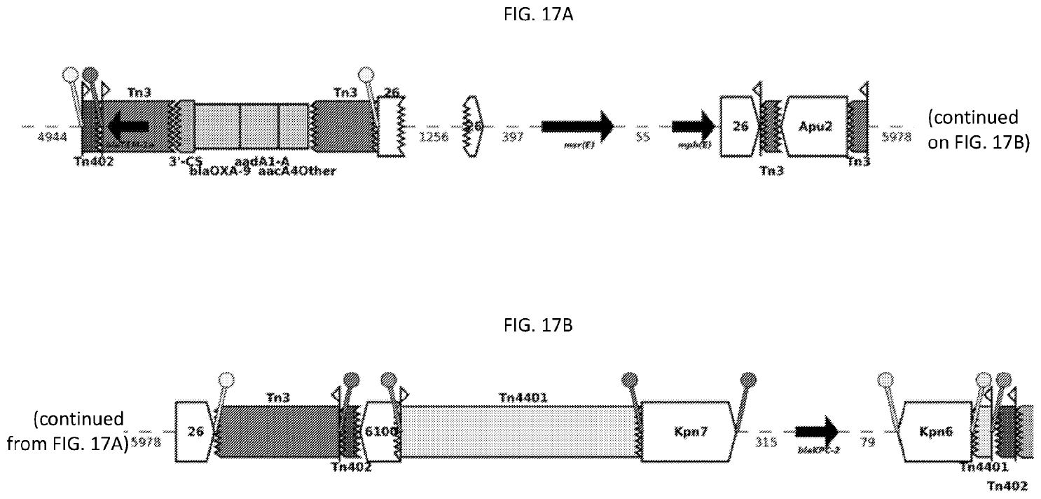

Additional annotation information is depicted graphically in an annotation image as shown in FIGS. 17A and 17B.

TABLE 1
CP011639 (Serratia marcescens) annotation.

```
  1. nil-match ↔ (1..4944 [4944])
  2. Direct Repeat (CGATG (4945..4949 [5]))
  3. Tn402 (Tn; 229603) [symbol]   (4950..5029 [80])
```

● IRt (IR; 44) → (4950..4974 [25])

```
  4. Direct Repeat (TATCA (5025..5029 [5]))
  5. Tn3 (Tn; 90) [symbol]   (5030..6538 [1509])
```

1. IR (IR; 231939) → (5030..5067 [38])
2. blaTEM-1a (R_gene; 32) ← (5177..6037 [861])

```
  6. 3′-CS (region; 1) [symbol]   (6539..6649 [111])
  7. CassArray ← (6650..9057 [2408])
```

1. blaOXA-9 (cassette; 231253) ← (6650..7606 [957])
2. aadA1-A (cassette; 231836) ← (7607..8418 [812])
3. aacA4Other (cassette; 229656) ← (8419..9057 [639])

```
  8. nil-match ↔ (9058..9065 [8])
  9. Tn3 (Tn; 90) [symbol]   (9066..27436 [18371])
```

1. Interruption → (10453..24872 [14420])

1. Direct Repeat (TATTATTC (10453..10460 [8]))
2. Inserted Unit → (10461..24864 [14404])

▪ Composite Transposon → (10461..24864 [14404])

1. IS26 (IS; 61) → (10461..12543 [2083])

1. Interruption ↔ (11029..12284 [1256])

▪ nil-match ↔ (11029..12284 [1256])

```
  2. nil-match ↔ (12544..12940 [397])
  3. mar(E) (R_gene; 231893) → (12941..14416 [1476])
  4. nil-match ↔ (14417..14471 [55])
  5. mph(E) (R_gene; 231892) → (14472..15356 [885])
  6. nil-match ↔ (15357..15376 [20])
  7. IS26 (IS; 229990) → (15377..16196 [820])
  8. nil-match ↔ (16197..16198 [2])
  9. IR (IR; 234492) ← (16199..16236 [38])
10. Tn3 (Tn; 90) [symbol]   (16237..18066 [1830])
```

1. Interruption ← (16438..17826 [1389])

▪ ISApu2 (IS; 229991) ← (16438..17826 [1389])
▪ IR (IR; 231939) ← (18029..18066 [38])

11. nil-match ↔ (18067..24044 [5978])
12. IS26 (IS; 61)→(24045..24864 [820])

3. Direct Repeat (TATTATTC (24865..24872 [8]))
▪ IR (IR; 231939) ← (27399..27436 [38])

10. Direct Repeat (TATCA (27437..27441 [5]))
11. Tn402 (Tn; 229603) [symbol] (27437..27513 [77])
12. IS6100 (IS; 64) [symbol] (27514..28382 [869])
13. Direct Repeat (TTTTT (28378..28382 [5]))
14. Tn4401 (Tn; 230162) [symbol] (28383..33309 [4927])

● Tn4401 IRL (IR; 231949) → (28383..28420 [38])

15. Direct Repeat (CCG(33307..33309 [3]))
16. Inserted Unit → (33310..35265 [1956])

● ISKpn7 (IS; 229917) → 33310..35265 [1956])

17. Direct Repeat (CCG (35266..35268 [3]))
18. nil-match ↔ (35269..35583 [315])
19. blaKPC-2 (R_gene; 231688) → (35584..36465 [882])
20. nil-match ↔ (36466..36544 [79])
21. Direct Repeat (TA (36545..36546 [2]))
22. Inserted Unit ← (36547..38086 [1540])

● ISKpn6 (IS; 229916) ← (36547..38086 [1540])

23. Direct Repeat (TA (38087..38088 [2]))
24. Tn4401 (Tn; 230162) [symbol] (38087..38388 [302])

● Tn4401 IRR (IR; 231950) ← (38351..38388 [38])

25. Direct Repeat (TTTTT (38389..38393 [5]))
26. nil-match ↔ (38394..38404 [11])
27. class 1 In/Tn ← (38405..43443 [5039])

● Tn402 (Tn; 229603) [symbol] (38405..38527 [123])

▪ IRt (IR; 44) ← (38503..38527 [25])

● CS+CA ← (38528..43443 [4916])

1. 3′-CS (region; 1) [symbol] (38528..40766 [2239])

▪ sul1 (R_gene; 78) ← (39478..40317 [840])

2. CassArray ← (40767..42091 [1325])

1. gouD (cassette; 203) ← (40767..41086 [320])
2. dfrA5 (cassette; 134) ← (41087..41654 [568])
3. gcu16 (cassette; 296) ← (41655..42091 [437])

3. 5′-CS (region; 7) ← (42092..43443 [1352])

▪ IRi (IR; 43) ← (43419..43443 [25])

28. Direct Repeat (CGATG (43444..43448 [5]))
29. nil-match ↔ (43449..69158 [25710])

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-102 are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A computer-implemented method for annotating a query nucleic acid sequence, the method comprising the following steps performed by one or more computer processors:
   receiving a query nucleic acid sequence, wherein the query nucleic acid sequence is a sequence or segment thereof of a nucleic acid obtained from a sample obtained from a defined physical location;
   accessing a relational database comprising a plurality of exemplar genetic elements and the following fields associated with each exemplar genetic element:
      one or more identifying fields,
      an exemplar nucleic acid sequence for the exemplar genetic element or an identifier of the exemplar nucleic acid sequence,
      a minimum identity match criterion or identifier thereof, and
      an identifier for a matching algorithm;
   receiving a selection of one or more of the exemplar genetic elements;
   for each of the selected one or more exemplar genetic elements, applying a corresponding matching algorithm identified in the identifier for a matching algorithm field to compare the query nucleic acid sequence with the exemplar nucleic acid sequence for the selected exemplar genetic element;
   for each of the selected one or more exemplar genetic elements, identifying whether results of the corresponding matching algorithm meet the minimum identity match criterion corresponding to the selected exemplar genetic element to provide a matched genetic element;
   for each matched genetic element, identifying whether constraints, if any, identified in the constraints identifier field corresponding to the selected exemplar genetic element have been met; and
   for one or more of the matched genetic elements without constraints and/or where the constraints corresponding to the selected exemplar genetic element have been met, annotating the query nucleic acid sequence with identifying information for the selected exemplar genetic element corresponding to the matched genetic element.
2. The method of 1, wherein the defined physical location is in a clinical setting.
3. The method of 2, wherein the clinical setting is an emergency room, an intensive care unit, an operating room, a hospital ward, or a combination thereof.
4. The method of any one of 1-3, wherein the query nucleic acid sequence is a sequence or segment thereof of a nucleic acid obtained from a bodily fluid.
5. The method of 4, wherein the bodily fluid is blood, saliva, sputum, feces, urine, or a combination thereof.
6. The method of any one of 1-5, wherein two or more matched genetic elements are provided that match to the same segment of the query nucleic acid sequence.
7. The method of 6, wherein when the two or more matched genetic elements that match to the same segment of the query nucleic acid sequence are of a different type, the identifying information for two or more selected exemplar genetic elements corresponding to the two or more matched genetic elements is used to annotate the same segment of the query nucleic acid sequence.
8. The method of 6, wherein when the two or more matched genetic elements that match to the same segment of the query nucleic acid sequence are non-overlapping, identifying information for two or more selected exemplar genetic elements corresponding to the two or more matched genetic elements is used to annotate the same segment of the query nucleic acid sequence.
9. The method of 6, wherein when the two or more matched genetic elements that match to the same segment of the query nucleic acid sequence have different calculated matching algorithm scores, identifying information for the selected exemplar genetic element corresponding to the matched genetic element with the highest calculated matching algorithm score is used to annotate the segment of the query nucleic acid sequence.
10. The method of 9, wherein the calculated matching algorithm scores indicate the level of match between the segment of the query nucleic acid sequence and the two or more matched genetic elements.
11. The method of any one of 1-10, wherein the query nucleic acid sequence is annotated with identifying information for two or more selected exemplar genetic elements corresponding to two or more matched genetic elements.
12. The method of 11, wherein the exemplar nucleic acid sequences for the two or more selected exemplar genetic elements corresponding to two or more matched genetic elements do not overlap.
13. The method of 11 or 12, further comprising identifying within the query nucleic acid sequence a gap sequence that is not annotated.
14. The method of 13, further comprising annotating the gap sequence by matching the gap sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the gap sequence with the exemplar nucleic acid sequence for the exemplar genetic element.
15. The method of 13, wherein the gap sequence comprises a truncated sequence of an exemplar nucleic acid sequence of an exemplar genetic element.
16. The method of 15, wherein the truncated sequence does not meet the minimum identity match criterion associated with the exemplar nucleic acid sequence of the exemplar genetic element.
17. The method of 15 or 16, wherein the nucleic acid sequence of the truncated sequence overlaps with a second exemplar nucleic acid sequence of a second exemplar genetic element.
18. The method of any one of 15-17, further comprising annotating the gap sequence by:
   expanding an end of the truncated sequence by one or more nucleotides to provide an expanded truncated sequence; and
   annotating the expanded truncated sequence by matching the expanded truncated sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the expanded truncated sequence with the exemplar nucleic acid sequence for the exemplar genetic element.
19. The method of any one of 1-18, wherein the minimum identity match criterion is a sequence identity of from about 50% to about 100% between the query nucleic acid sequence or a segment thereof and the exemplar nucleic acid sequence for a selected exemplar genetic element.
20. The method of any one of 1-19, wherein the corresponding matching algorithm for one or more of the one or more selected exemplar genetic elements is a Strict Match algorithm, a BLAST algorithm, a FASTA algorithm, a Smith-Waterman algorithm, a RegEx algorithm, or a combination thereof.
21. The method of any one of 1-20, wherein the relational database further comprises one or more of the following fields associated with each exemplar genetic element: a directional identifier, a completeness identifier, a direct repeats identifier, and a constraints identifier.
22. The method of any one of 1-21, wherein the relational database further comprises an alert field associated with each exemplar genetic element, wherein the alert field indicates whether the exemplar genetic element associated with the alert field corresponds with a matched genetic element.
23. The method of 21, wherein one or more of the selected one or more exemplar genetic elements has a corresponding constraint in the constraints identifier field corresponding to the selected exemplar genetic element.
24. The method of any one of 21-23, wherein the constraint comprises an open reading frame constraint, a specific nucleotide constraint, a length constraint, or a combination thereof.
25. The method of any one of 1-24, wherein one or more of the selected one or more exemplar genetic elements comprises a direct repeat.
26. The method of 25, further comprising determining whether the query nucleic acid comprises a direct repeat and annotating the query nucleic acid sequence with a direct repeats identifier when present.
27. The method of any one of 1-26, wherein the method for annotating a query nucleic acid sequence is performed on two or more computer processors operating in parallel.
28. The method of any one of 1-27, further comprising annotating an assembly of annotations made to the query nucleic acid sequence according to the method.
29. The method of 28, wherein annotating the assembly of annotations comprises:
   arranging a sequence for a first matched genetic element and a sequence for a second matched genetic element into a series of sequences for matched genetic elements; and
   processing the series of sequences for matched genetic elements using a parsing algorithm according to a predetermined set of parsing rules.
30. The method of 29, wherein when the sequence for the first matched genetic element is completely overlapped by the sequence for the second matched genetic element, the annotation for the first matched genetic element is removed from the assembly.
31. The method of 29 or 30, wherein the predetermined set of parsing rules allows for the identification of a mobile element.
32. The method of any one of 1-31, further comprising generating a readable representation of the annotated query nucleic acid sequence using a tree visualization method.
33. The method of any one of 1-32, further comprising generating a machine-readable representation of the annotated query nucleic acid sequence.
34. The method of any one of 1-33, further comprising generating a graphical representation of the annotated query nucleic acid sequence.
35. The method of any one of 32-34, wherein the readable representation, the machine-readable representation, and or the graphical representation of the annotated query nucleic acid sequence is stored in one or more databases.
36. The method of any one of 32-35, further comprising displaying a representation of the annotated query nucleic acid sequence on a client device.
37. The method of any one of 1-36, wherein the query nucleic acid sequence is a sequence or segment thereof of a nucleic acid obtained from an environmental sample from a first defined physical location at a first time point, and wherein the steps of the method are repeated for a second query nucleic acid sequence, wherein the second query nucleic acid sequence is a sequence or segment thereof of a nucleic acid obtained from an environmental sample from the first defined physical location at a second time point.
38. The method of any one of 1-37, wherein the relational database comprises a directional identifier field, and wherein the value for the directional identifier field for the selected exemplar genetic element corresponding to the matched genetic element indicates whether the direction of the corresponding exemplar nucleic acid sequence should be noted in the corresponding annotation of the query nucleic acid sequence.
39. The method of any one of 1-38, wherein the relational database comprises a completeness identifier field, and wherein the value for the completeness identifier field for the selected exemplar genetic element corresponding to the matched genetic element indicates whether the exemplar nucleic acid sequence for the exemplar genetic element is a complete or incomplete sequence for the selected exemplar genetic element.
40. The method of any one of 1-39, wherein the relational database comprises a direct repeats identifier field, and wherein the value for the direct repeats identifier field for the selected exemplar genetic element corresponding to the matched genetic element indicates whether the exemplar nucleic acid sequence for the exemplar genetic element includes direct repeats.
41. The method of any one of 1-40, wherein one or more of the exemplar genetic elements is an antibiotic resistance gene or a portion thereof.
42. A method of monitoring the genetic material of a population of organisms in a defined physical location, the method comprising: obtaining nucleic acid sequences from a representative sample of the population of organisms from the defined physical location at one or more time points; annotating nucleic acid sequences from each of the representative samples according to the method of any one of 1-41; and calculating a frequency of occurrence of a genetic element of interest in the population of organisms based on the annotation.
43. The method of 42, wherein the method comprises:
   obtaining nucleic acid sequences from a representative sample of the population of organisms from the defined physical location at two or more time points; and
   comparing the frequency of occurrence of the genetic element of interest in the population at a first time point to the frequency of occurrence of the genetic element of interest in the population at a second time point.
44. A method of monitoring the genetic material of a population of organisms in a defined physical location, the method comprising:
   collecting a representative sample of the population of organisms from the defined physical location at one or more time points;
   obtaining nucleic acid sequences from each of the representative samples;
   annotating the nucleic acid sequences according to the method of any one of 1-41; and
   calculating a frequency of occurrence of a genetic element of interest in the population of organisms based on the annotation.
45. The method of 44, wherein the method comprises:
   collecting the representative sample of the population of organisms from the defined physical location at two or more time points; and
   comparing the frequency of occurrence of the genetic element of interest in the population at a first time point to the frequency of occurrence of the genetic element of interest in the population at a second time point.
46. A method of monitoring the genetic material of a population of organisms in a defined physical location, the method comprising:
   collecting a representative sample of the population of organisms from the defined physical location at one or more time points;
   obtaining nucleic acid sequences from each of the representative samples;
   annotating the nucleic acid sequences by matching the nucleic acid sequences against a plurality of genetic elements in a relational database; and
   calculating a frequency of occurrence of a genetic element of interest in the population based on the annotation.
47. The method of 46, wherein the method comprises:
   collecting the representative sample of the population of organisms from the defined physical location at two or more time points; and
   comparing the frequency of occurrence of the genetic element of interest in the population at a first time point to the frequency of occurrence of the genetic element of interest in the population at a second, later time point.
48. The method of any one of 42-47, wherein the genetic element of interest is an antibiotic resistance gene.
49. The method of 48, wherein an increase in the frequency of occurrence of the antibiotic resistance gene at the second time point relative to the first time point indicates that the population of organisms in the defined physical location is exhibiting an increase in antibiotic resistance.
50. The method of any one of 46-49, wherein the two or more time points occur daily.
51. The method of any one of 46-49, wherein the two or more time points occur weekly.
52. The method of any one of 42-51, wherein the genetic element of interest is an antibiotic resistance gene and the method further comprises generating a report showing the frequency of occurrence of the antibiotic resistance gene or a graphical representation thereof.
53. The method of 52, wherein the report shows a trend in frequency of occurrence of the antibiotic resistance gene over time.
54. The method of any one of 48-53, comprising recommending a change in antibiotic use in the defined physical location based on the calculated frequency of occurrence of the antibiotic resistance gene or a change in the frequency of occurrence of the antibiotic resistance gene over time.
55. A method for obtaining an annotated nucleic acid sequence, the method comprising
   inputting a query nucleic acid sequence via a client device over a network connection to a server device, wherein the server device performs the method of any one of 1-41 to provide an annotated nucleic acid sequence; and
   receiving at the client device a representation of the annotated nucleic acid sequence.
56. A non-transitory computer-readable recording medium for annotating a query nucleic acid sequence, the non-transitory computer-readable recording medium comprising instructions, which, when executed by one or more processors, cause the one or more processors to perform a method for annotating a query nucleic acid sequence according to any one of 1-41.
57. A non-transitory computer-readable recording medium for annotating a query nucleic acid sequence, the non-transitory computer-readable recording medium comprising instructions, which, when executed by one or more processors, cause the one or more processors to:
   receive a query nucleic acid sequence, wherein the query nucleic acid sequence is a sequence or segment thereof of a nucleic acid obtained from a sample obtained from a defined physical location;
   access a relational database comprising a plurality of exemplar genetic elements and the following fields associated with each exemplar genetic element:
      one or more identifying fields,
      an exemplar nucleic acid sequence for the exemplar genetic element or an identifier of the exemplar nucleic acid sequence,
      a minimum identity match criterion or identifier thereof, and
      an identifier for a matching algorithm;
   receive a selection of one or more of the exemplar genetic elements;
   for each of the selected one or more exemplar genetic elements, apply a corresponding matching algorithm identified in the identifier for a matching algorithm field to compare the query nucleic acid sequence with the exemplar nucleic acid sequence for the selected exemplar genetic element;
   for each of the selected one or more exemplar genetic elements, identify whether results of the corresponding matching algorithm meet the minimum identity match criterion corresponding to the selected exemplar genetic element to provide a matched genetic element;
   for each matched genetic element, identify whether constraints, if any, identified in the constraints identifier field corresponding to the selected exemplar genetic element have been met; and
   for one or more of the matched genetic elements without constraints and/or where the constraints corresponding to the selected exemplar genetic element have been met, annotate the query nucleic acid sequence with identifying information for the selected exemplar genetic element corresponding to the matched genetic element.
58. The non-transitory recording medium of 57, wherein the defined physical location is in a clinical setting.
59. The non-transitory recording medium of 58, wherein the clinical setting is an emergency room, an intensive care unit, an operating room, a hospital ward, or a combination thereof.
60. The non-transitory recording medium of any one of 57-59, wherein the query nucleic acid sequence is a sequence or segment thereof of a nucleic acid obtained from a bodily fluid.
61. The non-transitory recording medium of 60, wherein bodily fluid is blood, saliva, sputum, feces, urine, or a combination thereof.
62. The non-transitory recording medium of any one of 57-61, wherein two or more matched genetic elements are provided that match to the same segment of the query nucleic acid sequence.
63. The non-transitory recording medium of 62, wherein when the two or more matched genetic elements that match to the same segment of the query nucleic acid sequence are of a different type, the identifying information for two or more selected exemplar genetic elements corresponding to the two or more matched genetic elements is used to annotate the same segment of the query nucleic acid sequence.
64. The non-transitory recording medium of 62, wherein when the two or more matched genetic elements that match to the same segment of the query nucleic acid sequence are non-overlapping, identifying information for two or more selected exemplar genetic elements corresponding to the two or more matched genetic elements is used to annotate the same segment of the query nucleic acid sequence.
65. The non-transitory recording medium of 62, wherein when the two or more matched genetic elements that match to the same segment of the query nucleic acid sequence have different calculated matching algorithm scores, identifying information for the selected exemplar genetic element corresponding to the matched genetic element with the highest calculated matching algorithm score is used to annotate the segment of the query nucleic acid sequence.
66. The non-transitory recording medium of 65, wherein the calculated matching algorithm scores indicate the level of match between the segment of the query nucleic acid sequence and the two or more matched genetic elements.
67. The non-transitory recording medium of any one of 57-66, wherein the query nucleic acid sequence is annotated with identifying information for two or more selected exemplar genetic elements corresponding to two or more matched genetic elements.
68. The non-transitory recording medium of 67, wherein the exemplar nucleic acid sequences for the two or more selected exemplar genetic elements corresponding to two or more matched genetic elements do not overlap.
69. The non-transitory recording medium of 67 or 68, further comprising instructions, which, when executed by the one or more processors, cause the one or more processors to identify within the query nucleic acid sequence a gap sequence that is not annotated.
70. The non-transitory recording medium of 69, further comprising instructions, which, when executed by the one or more processors, cause the one or more processors to annotate the gap sequence by matching the gap sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the gap sequence with the exemplar nucleic acid sequence for the exemplar genetic element.
71. The non-transitory recording medium of 69, wherein the gap sequence comprises a truncated sequence of an exemplar nucleic acid sequence.
72. The non-transitory recording medium of 71, wherein the truncated sequence does not meet the minimum identity match criterion associated with the exemplar nucleic acid sequence.
73. The non-transitory recording medium of 71 or 72, wherein the exemplar nucleic acid sequence of the truncated sequence overlaps with a second exemplar nucleic acid sequence.
74. The non-transitory recording medium of any one of 71-73, further comprising instructions, which, when executed by the one or more processors, cause the one or more processors to annotate the gap sequence by;
   expanding an end of the truncated sequence by one or more nucleotides to provide an expanded truncated sequence; and
   annotating the expanded truncated sequence by matching the expanded truncated sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the expanded truncated sequence with the exemplar nucleic acid sequence for the exemplar genetic element.
75. The non-transitory recording medium of any one of 57-74, wherein the minimum identity match criterion is a sequence identity of from about 50% to about 100% between the query nucleic acid sequence or a segment thereof and the exemplar nucleic acid sequence for a selected exemplar genetic element.
76. The non-transitory recording medium of any one of 57-75, wherein the corresponding matching algorithm for one or more of the one or more selected exemplar genetic elements is a Strict Match algorithm, a BLAST algorithm, a FASTA algorithm, a Smith-Waterman algorithm, a RegEx algorithm, or a combination thereof
77. The non-transitory recording medium of any one of 57-76, wherein the relational database further comprises one or more of the following fields associated with each exemplar genetic element: a directional identifier, a completeness identifier, a direct repeats identifier, and a constraints identifier.
78. The non-transitory recording medium of any one of 57-77, wherein the relational database further comprises an alert field associated with each exemplar genetic element, wherein the alert field indicates whether the exemplar genetic element associated with the alert field corresponds with a matched genetic element.
79. The non-transitory recording medium of 77, wherein one or more of the selected one or more exemplar genetic elements has a corresponding constraint in the constraints identifier field corresponding to the selected exemplar genetic element.
80. The non-transitory recording medium of any one of 77-79, wherein the constraint comprises an open reading frame constraint, a specific nucleotide constraint, a length constraint, or a combination thereof
81. The non-transitory recording medium of any one of 57-80, wherein one or more of the selected one or more exemplar genetic elements comprises a direct repeat.
82. The non-transitory recording medium of 81, further comprising instructions, which, when executed by the one or more processors, cause the one or more processors to determine whether the query nucleic acid comprises a direct repeat, and annotate the query nucleic acid sequence with a direct repeats identifier when present.
83. The non-transitory recording medium of any one of 57-82, wherein the instructions are executed by two or more computer processors operating in parallel.
84. The non-transitory recording medium of any one of 57-83, further comprising instructions, which, when executed by the one or more processors, cause the one or more processors to annotate an assembly of annotations made to the query nucleic acid sequence according to the method.
85. The non-transitory recording medium of 84, wherein annotating the assembly of annotations comprises instructions, which, when executed by the one or more processors, cause the one or more processors to:
   arrange a sequence for a first matched genetic element and a sequence for a second matched genetic element into a series of sequences for matched genetic elements; and
   process the series of sequences for matched genetic elements using a parsing algorithm according to a predetermined set of parsing rules.
86. The non-transitory recording medium of 85, wherein when the sequence for the first matched genetic element is completely overlapped by the sequence for the second matched genetic element, the annotation for the first matched genetic element is removed from the assembly.
87. The non-transitory recording medium of 85 or 86, wherein the predetermined set of parsing rules allows for the identification of a mobile element.
88. The non-transitory recording medium of any one of 57-87, further comprising instructions, which, when executed by the one or more processors, cause the one or more processors to generate a readable representation of the annotated query nucleic acid sequence using a tree visualization method.
89. The non-transitory recording medium of any one of 57-88, further comprising instructions, which, when executed by the one or more processors, cause the one or more processors to generate a machine-readable representation of the annotated query nucleic acid sequence.
90. The non-transitory recording medium of any one of 57-89, further comprising instructions, which, when executed by the one or more processors, cause the one or more processors to generate a graphical representation of the annotated query nucleic acid sequence.
91. The non-transitory recording medium of any one of 88-90, wherein the readable representation, the machine-readable representation, and or the graphical representation of the annotated query nucleic acid sequence is stored in one or more databases.
92. The method of any one of 88-91, further comprising instructions, which, when executed by the one or more processors, cause the one or more processors to display a representation of the annotated query nucleic acid sequence on a client device.
93. The non-transitory recording medium of any one of 57-92, wherein the query nucleic acid sequence is a sequence or segment thereof of a nucleic acid obtained from an environmental sample from a first defined physical location at a first time point, and wherein the steps of the method are repeated for a second query nucleic acid sequence, wherein the second query nucleic acid sequence is a sequence or segment thereof of a nucleic acid obtained from an environmental sample from the first defined physical location at a second time point.
94. The non-transitory recording medium of any one of 57-93, wherein the relational database comprises a directional identifier field, and wherein the value for the directional identifier field for the selected exemplar genetic element corresponding to the matched genetic element indicates whether the direction of the corresponding exemplar nucleic acid sequence should be noted in the corresponding annotation of the query nucleic acid sequence.
95. The non-transitory recording medium of any one of 57-94, wherein the relational database comprises a completeness identifier field, and wherein the value for the completeness identifier field for the selected exemplar genetic element corresponding to the matched genetic element indicates whether the exemplar nucleic acid sequence for the exemplar genetic element is a complete or incomplete sequence for the selected exemplar genetic element.
96. The non-transitory recording medium of any one of 57-95, wherein the relational database comprises a direct repeats identifier field, and wherein the value for the direct repeats identifier field for the selected exemplar genetic element corresponding to the matched genetic element indicates whether the exemplar nucleic acid sequence for the exemplar genetic element includes direct repeats.
97. The non-transitory recording medium of any one of 57-96, wherein one or more of the exemplar genetic elements is an antibiotic resistance gene or a portion thereof 98. A system for annotating a query nucleic acid sequence, the system comprising:
   a communication module comprising an input manager for receiving the query nucleic acid sequence from a user;
   an output manager for communicating output to a user; and
   a non-transitory computer-readable recording medium according to any one of 57-97.
99. The system of 98 further comprising:
   an alert module for alerting the user when a specific genetic element has been annotated.
100. The system of 98 or 99, wherein the user is in a remote location.
101. The system of 99 or 100, wherein the user is alerted via an electronic mail, a short message service, a mobile application notification, or a combination thereof.
102. A non-limiting aspect of the disclosure as described in any one of 1-101 above, adapted for annotation of a polypeptide sequence.

What is claimed is:

1. A computer-implemented method for annotating a query nucleic acid sequence, the method comprising the following steps performed by one or more computer processors:
receiving a query nucleic acid sequence, wherein the query nucleic acid sequence is a sequence or segment thereof of a nucleic acid obtained from a sample obtained from a defined physical location;
accessing a relational database comprising a plurality of exemplar genetic elements and the following fields associated with each exemplar genetic element:
one or more identifying fields,
an exemplar nucleic acid sequence for the exemplar genetic element or an identifier of the exemplar nucleic acid sequence,
a minimum identity match criterion or identifier thereof, and
an identifier for a matching algorithm;
receiving a selection of one or more of the exemplar genetic elements;
for each of the selected one or more exemplar genetic elements, applying a corresponding matching algorithm identified in the identifier for a matching algorithm field to compare the query nucleic acid sequence with the exemplar nucleic acid sequence for the selected exemplar genetic element;
for each of the selected one or more exemplar genetic elements, identifying whether results of the corresponding matching algorithm meet the minimum identity match criterion corresponding to the selected exemplar genetic element to provide a matched genetic element;
for each matched genetic element, identifying whether constraints, if any, identified in the constraints identifier field corresponding to the selected exemplar genetic element have been met; and
for one or more of the matched genetic elements without constraints and/or where the constraints corresponding to the selected exemplar genetic element have been met, annotating the query nucleic acid sequence with identifying information for the selected exemplar genetic element corresponding to the matched genetic element.

2. The method of claim 1, wherein the defined physical location is in a clinical setting.

3. The method of claim 2, wherein the clinical setting is an emergency room, an intensive care unit, an operating room, a hospital ward, or a combination thereof.

4. The method of any one of claims 1-3, wherein the query nucleic acid sequence is a sequence or segment thereof of a nucleic acid obtained from a bodily fluid.

5. The method of claim 4, wherein the bodily fluid is blood, saliva, sputum, feces, urine, or a combination thereof.

6. The method of any one of claims 1-5, wherein two or more matched genetic elements are provided that match to the same segment of the query nucleic acid sequence.

7. The method of claim 6, wherein when the two or more matched genetic elements that match to the same segment of the query nucleic acid sequence are of a different type, the identifying information for two or more selected exemplar genetic elements corresponding to the two or more matched genetic elements is used to annotate the same segment of the query nucleic acid sequence.

8. The method of claim 6, wherein when the two or more matched genetic elements that match to the same segment of the query nucleic acid sequence are non-overlapping, identifying information for two or more selected exemplar genetic elements corresponding to the two or more matched genetic elements is used to annotate the same segment of the query nucleic acid sequence.

9. The method of claim 6, wherein when the two or more matched genetic elements that match to the same segment of the query nucleic acid sequence have different calculated matching algorithm scores, identifying information for the selected exemplar genetic element corresponding to the matched genetic element with the highest calculated matching algorithm score is used to annotate the segment of the query nucleic acid sequence.

10. The method of claim 9, wherein the calculated matching algorithm scores indicate the level of match between the segment of the query nucleic acid sequence and the two or more matched genetic elements.

11. The method of any one of claims 1-10, wherein the query nucleic acid sequence is annotated with identifying information for two or more selected exemplar genetic elements corresponding to two or more matched genetic elements.

12. The method of claim 11, wherein the exemplar nucleic acid sequences for the two or more selected exemplar genetic elements corresponding to two or more matched genetic elements do not overlap.

13. The method of claim 11 or 12, further comprising identifying within the query nucleic acid sequence a gap sequence that is not annotated.

14. The method of claim 13, further comprising annotating the gap sequence by matching the gap sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the gap sequence with the exemplar nucleic acid sequence for the exemplar genetic element.

15. The method of claim 13, wherein the gap sequence comprises a truncated sequence of an exemplar nucleic acid sequence of an exemplar genetic element.

16. The method of claim 15, wherein the truncated sequence does not meet the minimum identity match criterion associated with the exemplar nucleic acid sequence of the exemplar genetic element.

17. The method of claim 15 or 16, wherein the nucleic acid sequence of the truncated sequence overlaps with a second exemplar nucleic acid sequence of a second exemplar genetic element.

18. The method of any one of claims 15-17, further comprising annotating the gap sequence by:
expanding an end of the truncated sequence by one or more nucleotides to provide an expanded truncated sequence; and
annotating the expanded truncated sequence by matching the expanded truncated sequence to the exemplar nucleic acid sequence for one or more of the exemplar genetic elements in the relational database, wherein the matching comprises applying a corresponding matching algorithm identified in the identifier for a matching algorithm field for the exemplar genetic element to compare the expanded truncated sequence with the exemplar nucleic acid sequence for the exemplar genetic element.

19. The method of any one of claims 1-18, wherein the minimum identity match criterion is a sequence identity of from about 50% to about 100% between the query nucleic acid sequence or a segment thereof and the exemplar nucleic acid sequence for a selected exemplar genetic element.

20. The method of any one of claims 1-19, wherein the corresponding matching algorithm for one or more of the one or more selected exemplar genetic elements is a Strict Match algorithm, a BLAST algorithm, a FASTA algorithm, a Smith-Waterman algorithm, a RegEx algorithm, or a combination thereof.

21. The method of any one of claims 1-20, wherein the relational database further comprises one or more of the following fields associated with each exemplar genetic element: a directional identifier, a completeness identifier, a direct repeats identifier, and a constraints identifier.

22. The method of any one of claims 1-21, wherein the relational database further comprises an alert field associated with each exemplar genetic element, wherein the alert field indicates whether the exemplar genetic element associated with the alert field corresponds with a matched genetic element.

23. The method of claim 21, wherein one or more of the selected one or more exemplar genetic elements has a corresponding constraint in the constraints identifier field corresponding to the selected exemplar genetic element.

24. The method of any one of claims 21-23, wherein the constraint comprises an open reading frame constraint, a specific nucleotide constraint, a length constraint, or a combination thereof.

25. The method of any one of claims 1-24, wherein one or more of the selected one or more exemplar genetic elements comprises a direct repeat.

26. The method of claim 25, further comprising determining whether the query nucleic acid comprises a direct repeat and annotating the query nucleic acid sequence with a direct repeats identifier when present.

27. The method of any one of claims 1-26, wherein the method for annotating a query nucleic acid sequence is performed on two or more computer processors operating in parallel.

28. The method of any one of claims 1-27, further comprising annotating an assembly of annotations made to the query nucleic acid sequence according to the method.

29. The method of claim 28, wherein annotating the assembly of annotations comprises:
arranging a sequence for a first matched genetic element and a sequence for a second matched genetic element into a series of sequences for matched genetic elements; and
processing the series of sequences for matched genetic elements using a parsing algorithm according to a predetermined set of parsing rules.

30. The method of claim 29, wherein when the sequence for the first matched genetic element is completely overlapped by the sequence for the second matched genetic element, the annotation for the first matched genetic element is removed from the assembly.

31. The method of claim 29 or 30, wherein the predetermined set of parsing rules allows for the identification of a mobile element.

32. The method of any one of claims 1-31, further comprising generating a readable representation of the annotated query nucleic acid sequence using a tree visualization method.

33. The method of any one of claims 1-32, further comprising generating a machine-readable representation of the annotated query nucleic acid sequence.

34. The method of any one of claims 1-33, further comprising generating a graphical representation of the annotated query nucleic acid sequence.

35. The method of any one of claims 32-34, wherein the readable representation, the machine-readable representation, and or the graphical representation of the annotated query nucleic acid sequence is stored in one or more databases.

36. The method of any one of claims 32-35, further comprising displaying a representation of the annotated query nucleic acid sequence on a client device.

37. The method of any one of claims 1-36, wherein the query nucleic acid sequence is a sequence or segment thereof of a nucleic acid obtained from an environmental sample from a first defined physical location at a first time point, and wherein the steps of the method are repeated for a second query nucleic acid sequence, wherein the second query nucleic acid sequence is a sequence or segment thereof of a nucleic acid obtained from an environmental sample from the first defined physical location at a second time point.

38. The method of any one of claims 1-37, wherein the relational database comprises a directional identifier field, and wherein the value for the directional identifier field for the selected exemplar genetic element corresponding to the matched genetic element indicates whether the direction of the corresponding exemplar nucleic acid sequence should be noted in the corresponding annotation of the query nucleic acid sequence.

39. The method of any one of claims 1-38, wherein the relational database comprises a completeness identifier field, and wherein the value for the completeness identifier field for the selected exemplar genetic element corresponding to the matched genetic element indicates whether the exemplar nucleic acid sequence for the exemplar genetic element is a complete or incomplete sequence for the selected exemplar genetic element.

40. The method of any one of claims 1-39, wherein the relational database comprises a direct repeats identifier field, and wherein the value for the direct repeats identifier field for the selected exemplar genetic element corresponding to the matched genetic element indicates whether the exemplar nucleic acid sequence for the exemplar genetic element includes direct repeats.

41. The method of any one of claims 1-40, wherein one or more of the exemplar genetic elements is an antibiotic resistance gene or a portion thereof.

42. A method of monitoring the genetic material of a population of organisms in a defined physical location, the method comprising: obtaining nucleic acid sequences from a representative sample of the population of organisms from the defined physical location at one or more time points; annotating nucleic acid sequences from each of the representative samples according to the method of any one of claims 1-41; and calculating a frequency of occurrence of a genetic element of interest in the population of organisms based on the annotation.

43. The method of claim 42, wherein the method comprises:
obtaining nucleic acid sequences from a representative sample of the population of organisms from the defined physical location at two or more time points; and
comparing the frequency of occurrence of the genetic element of interest in the population at a first time point to the frequency of occurrence of the genetic element of interest in the population at a second time point.

44. A method of monitoring the genetic material of a population of organisms in a defined physical location, the method comprising:
collecting a representative sample of the population of organisms from the defined physical location at one or more time points;
obtaining nucleic acid sequences from each of the representative samples;
annotating the nucleic acid sequences according to the method of any one of claims 1-41; and
calculating a frequency of occurrence of a genetic element of interest in the population of organisms based on the annotation.

45. The method of claim 44, wherein the method comprises:
collecting the representative sample of the population of organisms from the defined physical location at two or more time points; and
comparing the frequency of occurrence of the genetic element of interest in the population at a first time point to the frequency of occurrence of the genetic element of interest in the population at a second time point.

46. A method of monitoring the genetic material of a population of organisms in a defined physical location, the method comprising:
collecting a representative sample of the population of organisms from the defined physical location at one or more time points;
obtaining nucleic acid sequences from each of the representative samples;
annotating the nucleic acid sequences by matching the nucleic acid sequences against a plurality of genetic elements in a relational database; and
calculating a frequency of occurrence of a genetic element of interest in the population based on the annotation.

47. The method of claim 46, wherein the method comprises:
collecting the representative sample of the population of organisms from the defined physical location at two or more time points; and
comparing the frequency of occurrence of the genetic element of interest in the population at a first time point to the frequency of occurrence of the genetic element of interest in the population at a second, later time point.

48. The method of any one of claims 42-47, wherein the genetic element of interest is an antibiotic resistance gene.

49. The method of claim 48, wherein an increase in the frequency of occurrence of the antibiotic resistance gene at the second time point relative to the first time point indicates that the population of organisms in the defined physical location is exhibiting an increase in antibiotic resistance.

50. The method of any one of claims 46-49, wherein the two or more time points occur daily.

51. The method of any one of claims 46-49, wherein the two or more time points occur weekly.

52. The method of any one of claims 42-51, wherein the genetic element of interest is an antibiotic resistance gene and the method further comprises generating a report showing the frequency of occurrence of the antibiotic resistance gene or a graphical representation thereof.

53. The method of claim 52, wherein the report shows a trend in frequency of occurrence of the antibiotic resistance gene over time.

54. The method of any one of claims 48-53, comprising recommending a change in antibiotic use in the defined physical location based on the calculated frequency of occurrence of the antibiotic resistance gene or a change in the frequency of occurrence of the antibiotic resistance gene over time.

55. A method for obtaining an annotated nucleic acid sequence, the method comprising
inputting a query nucleic acid sequence via a client device over a network connection to a server device, wherein the server device performs the method of any one of claims 1-41 to provide an annotated nucleic acid sequence; and
receiving at the client device a representation of the annotated nucleic acid sequence.

56. A non-transitory computer-readable recording medium for annotating a query nucleic acid sequence, the non-transitory computer-readable recording medium comprising instructions, which, when executed by one or more processors, cause the one or more processors to perform a method for annotating a query nucleic acid sequence according to any one of claims 1-41.
